(19) United States
(12) Patent Application Publication
(10) Pub. No.: US 2026/0259232 A1
(43) Pub. Date: Sep. 3, 2026

(54) A METHOD

(71) Applicant: OWLSTONE MEDICAL LIMITED, Cambridge (GB)

(72) Inventor: Giuseppe FERRANDINO, Cambridge (GB)

(21) Appl. No.: 19/127,010

(22) PCT Filed: Nov. 7, 2023
(86) PCT No.: PCT/GB2023/052909

§ 371 Date: May 2, 2025

(30) Foreign Application Priority Data
Nov. 7, 2022 (GB) 2216545.0

(51) Int. Cl. G01N 33/98; G01N 33/58

(52) U.S. Cl. CPC G01N 33/98; G01N 33/58; G01N 2458/15; G01N 2800/085; G01N 2800/52

(57) ABSTRACT

A method for identifying a patient having reduced liver-function or a patient at risk of reduced liver function that will respond to therapy with an antibiotic and/or antifungal agent comprising measuring the concentration of ethanol, one or more sugar fermentation product, such as 2,3-butanediol, and/or a direct ethanol biomarker in a biological sample from said patient, wherein the concentration of ethanol, one or more sugar fermentation product, such as 2,3-butanediol, and/or a direct ethanol biomarker is indicative that said patient will respond to therapy with an antibiotic and/or antifungal agent.

### FIELD

The invention relates to methods for identifying a patient having reduced liver-function or a patient at risk of reduced liver function that will respond to therapy with an antibiotic and/or antifungal agent and related methods.

### BACKGROUND

Hepatic cirrhosis is scarring (fibrosis) of the liver, which is typically caused by long-term liver damage. Cirrhosis can be classified into four stages: 1) steatosis, 2) scarring or fibrosis, 3) cirrhosis and 4) liver failure, advanced liver disease or hepatic failure. A patient may develop liver cirrhosis as a result of factors including, but not limited to, excessive alcohol consumption, viral hepatitis, autoimmune hepatitis, etc.

Nonalcoholic Fatty Liver Disease (NAFLD) encompasses an entire histologic spectrum ranging from simple, benign hepatic steatosis to non-alcoholic steatohepatitis (NASH) characterized by lipid accumulation, inflammation, hepatocyte ballooning, and varying degrees of fibrosis. NASH may progress to cirrhosis or hepatocellular carcinoma (HCC). NASH, unlike non-alcoholic fatty liver (NAFL), has the greatest potential to progress to cirrhosis, liver failure, and liver cancer. The prevalence of NAFLD is increasing and is linked to the increase in cases of obesity. Despite increasing awareness of obesity-related liver disease, the pathogenesis of NAFLD and NASH remains poorly understood.

Based on the prevalence of NAFLD, it is anticipated that NASH-induced cirrhosis will become the most common indication for liver transplantation in the future. Differentiating NASH from simple steatosis is important for the clinical management of NAFLD patients and to reduce mortality (Chen et al, Radiology. 2011 June; 259(3): 749-756).

There is a growing body of evidence linking the gut microbiome to a role in the pathogenesis and progression of liver disease, such as NASH (Forlano et al, Int. J. Mol. Sci., 2022, 23, 8307). For example, preliminary studies have shown that NASH is associated with small intestinal bacterial overgrowth in human subjects (Wigg et al, Gut, 2001, 48, 206-211). Further studies have shown an increase in the abundance of Proteobacteria and Firmicutes, as well as a reduction in Bacteroidetes and Prevotellaceae, in the gut microbiome of patients with NAFLD/NASH compared to healthy controls (Hoyles et al, Nat. Med. 2018, 24, 1070-1080; on-Wisnewsky, J. et al, Nat. Rev. Gastroenterol. Hepatol. 2020, 17, 279-297; Lanthier et al, Sci. Rep. 2021, 11, 659; and Astbury et al, Gut Microbes 2020, 11, 569-580).

The use of antibiotics for the treatment of liver disease, with the aim of modifying the gut microbiome, has also been investigated. For example, rifaximin and solithromycin have been investigated in human studies for treating NAFLD (Ponziani et al, Dig. Dis. 2016, 34, 269-278; and Cobbold et al, Hepatol. Res. 2018, 48, 69-77).

Thus, there is a need to provide efficacious treatments of liver disease, such as NAFLD and NASH, as well as biomarkers that are predictive for a response to treatment. The present invention is aimed at addressing one or more of the aforementioned needs.

### SUMMARY

In a first aspect there is provided a method for identifying a patient having reduced liver-function or a patient at risk of reduced liver function that will respond to therapy with an antibiotic and/or antifungal agent comprising measuring the concentration of ethanol, one or more sugar fermentation product, such as 2,3-butanediol, and/or a direct ethanol biomarker in a biological sample from said patient, wherein the concentration of ethanol, one or more sugar fermentation product, such as 2,3-butanediol, and/or a direct ethanol biomarker is indicative that said patient will respond to therapy with an antibiotic and/or antifungal agent.

Without being bound by theory, it is thought that some patients having reduced liver function may have an overgrowth of bacteria and/or yeast in the gut, mouth and/or urinary tract. Such an overgrowth of bacteria and/or yeast may result in increased levels of toxic compounds, such as ethanol, one or more sugar fermentation product, such as 2,3-butanediol, and/or a direct ethanol biomarker through fermentation. Patients having an overgrowth of bacteria and/or yeast in the gut, mouth and/or urinary tract as a result of reduced liver function may suitably respond to treatment with an antibiotic and/or antifungal agent. For example, and again without being bound by theory and by way of example only, it may be speculated that the increased abundance of Proteobacteria, a phylum which includes alcohol producing bacteria, in the gut microbiome of a patient that will respond to therapy with an antibiotic may contribute to an increased concentration of ethanol, one or more sugar fermentation product, such as 2,3-butanediol, and/or a direct ethanol biomarker in a biological sample obtained from said patient (compared to a patient that will not respond to therapy with an antibiotic).

For the avoidance of doubt, the gut microbiome refers to the microorganism community residing in the intestinal lumen, under physiologic and/or pathologic conditions, and includes the microorganisms as well as their genomes and the surrounding environmental conditions.

In one embodiment, the method may comprise the steps of:
a) measuring the concentration of ethanol, one or more sugar fermentation product, such as 2,3-butanediol, and/or a direct ethanol biomarker in a biological sample obtained from the patient; and
b) comparing the concentration to a reference value from a subject with reduced liver function that does not respond to therapy with an antibiotic and/or antifungal agent; and/or from a subject with reduced liver function that responds to therapy with an antibiotic and/or antifungal agent; and/or from a healthy subject.
   wherein if the reference value is from a subject that does not respond to therapy with an antibiotic and/or antifungal agent, then an increase in the concentration of ethanol, one or more sugar fermentation product, such as 2,3-butanediol, and/or a direct ethanol biomarker compared to the reference value is indicative that the patient will respond to therapy with an antibiotic and/or antifungal agent; or
   wherein if the reference value is from a subject that does respond to therapy with an antibiotic and/or antifungal agent, then the same or substantially the same or an increase in concentration of ethanol, one or more sugar fermentation product, such as 2,3-butanediol, and/or a direct ethanol biomarker is indicative that the subject will respond to therapy with an antibiotic and/or antifungal agent;
   wherein if the reference value is from a healthy subject then an increase in the concentration of ethanol, one or more sugar fermentation product, such as 2,3-butanediol, and/or a direct ethanol biomarker compared to the reference value is indicative that the patient will respond to therapy with an antibiotic and/or antifungal agent.

In one embodiment, the method may be for identifying a patient having reduced liver-function or a patient at risk of reduced liver function that will respond to therapy with an antibiotic, and/or antifungal agent wherein the concentration of ethanol, one or more sugar fermentation product, such as 2,3-butanediol, and/or a direct ethanol biomarker is indicative that said patient will respond to therapy with an antibiotic and/or antifungal agent.

In one embodiment, the method may comprise measuring the concentration of ethanol in a biological sample obtained from a patient.

In one embodiment, the patient may have been diagnosed with a liver disease.

In one embodiment, the patient may have non-alcoholic fatty liver disease (NAFLD), such as non-alcoholic steatohepatitis (NASH).

In one embodiment, the patient may not have been diagnosed with a liver disease; and wherein the patient is at risk of reduced liver function.

In one embodiment, the patient may have one or more risk factors selected from obesity, alcoholism, family history of liver disease, type 2 diabetes, dyslipidemia, and/or a genetic predisposition to a liver disease.

In one embodiment, the method may further comprise the step of identifying a patient in need of treatment with an antibiotic and/or antifungal agent.

In one embodiment, the method may further comprise administering an antibiotic and/or antifungal agent to said patient.

In one embodiment, the method may further comprise the step of obtaining a biological sample from the patient.

In one embodiment, the biological sample may be hair, blood, stool, urine and/or exhaled breath, for example blood, urine and/or exhaled breath.

In one embodiment, the biological sample may be exhaled breath.

In one embodiment, the method may further comprise administering a carbohydrate to the patient; wherein the step of administering a carbohydrate to the patient is carried out prior to the step of obtaining a biological sample from the patient.

In one embodiment, the carbohydrate may be labelled such that at least a portion of the ethanol, one or more sugar fermentation product, such as 2,3-butanediol, and/or a direct ethanol biomarker is labelled.

In one embodiment, the label may be 12C, 13C, 14C, 2H, 17O and/or 18O, such as 13C.

In one embodiment, the step of administering a carbohydrate to the patient may be carried out at least 10, such as 20, 30, 40, 50, 60, 90 or 120, 180 minutes prior to the step of obtaining a biological sample from the patient.

In one embodiment, the method may further comprise the step of administering an alcohol dehydrogenase (ADH) inhibitor to the patient; wherein the step of administering an ADH inhibitor to the patient is carried out prior to the step of obtaining a biological sample from the patient.

In one embodiment, where the method includes the step of administering a carbohydrate to a patient, the step of administering an ADH inhibitor to the patient may be carried out before and/or after the step of administering a carbohydrate to a patient.

In one embodiment, the biological sample may be blood; and a concentration of ethanol, one or more sugar fermentation product, such as 2,3-butanediol, and/or a direct ethanol biomarker of at least 20 mmol/L may be indicative that said patient will respond to therapy with an antibiotic and/or antifungal agent.

In one embodiment, the biological sample may be exhaled breath; and a concentration of ethanol, one or more sugar fermentation product, such as 2,3-butanediol, and/or a direct ethanol biomarker of at least 0.0025 mmol/L, such as at least 0.005 mmol/L, such as at least 0.01 mmol/L, may be indicative that said patient will respond to therapy with an antibiotic and/or antifungal agent.

In one embodiment, the ethanol, one or more sugar fermentation product, such as 2,3-butanediol, and/or a direct ethanol biomarker may be labelled; and the presence of labelled ethanol, one or more sugar fermentation product, such as 2,3-butanediol, and/or a direct ethanol biomarker in the biological sample from the patient may be indicative that said patient will respond to therapy with an antibiotic and/or antifungal agent.

In one embodiment, the antibiotic may comprise rifaximin; neomycin; metronidazole; flouroquinolones, such as norfloxacin and ciprofloxacin; cephalosporins, such as ceftriaxone and cefotaxime; trimethoprim-sulfamethoxazole; flucloxacillin; cephalothin; aztreonam; cabimicina; and combinations thereof.

In a further aspect there is provided a method of predicting a response to an antibiotic and/or antifungal agent therapy in a patient having reduced liver-function or at risk of reduced liver function comprising measuring the concentration of ethanol, one or more sugar fermentation product, such as 2,3-butanediol, and/or a direct ethanol biomarker in a biological sample from said patient, wherein the concentration of ethanol, one or more sugar fermentation product, such as 2,3-butanediol, and/or a direct ethanol biomarker is indicative of a response or non-response of a patient to therapy with an antibiotic and/or antifungal agent.

In one embodiment, the method may comprise the steps of:
a) measuring the concentration of ethanol, one or more sugar fermentation product, such as 2,3-butanediol, and/or a direct ethanol biomarker in a biological sample obtained from the patient; and
b) comparing the concentration to a reference value from a subject with reduced liver function that does not respond to therapy with an antibiotic and/or antifungal agent; and/or from a subject with reduced liver function that does respond to therapy with an antibiotic and/or antifungal agent; and/or from a healthy subject;
   wherein if the reference level is from a subject that does not respond to therapy with an antibiotic and/or antifungal agent, then an increase in the concentration of ethanol, one or more sugar fermentation product, such as 2,3-butanediol, and/or a direct ethanol biomarker compared to the reference value is indicative that the patient will respond to therapy with an antibiotic and/or antifungal agent; or
   wherein if the reference level is from a subject that does respond to therapy with an antibiotic and/or antifungal agent, then the same or substantially the same or an increase in concentration of ethanol, one or more sugar fermentation product, such as 2,3-butanediol, and/or a direct ethanol biomarker is indicative that the patient will respond to therapy with an antibiotic and/or antifungal agent;
   wherein if the reference value is from a healthy subject then an increase in the concentration of ethanol, one or more sugar fermentation product, such as 2,3-butanediol, and/or a direct ethanol biomarker compared to the reference value is indicative that the patient will respond to therapy with an antibiotic and/or antifungal agent.

In one embodiment, the method may comprise the step of predicting a response to therapy with an antibiotic and/or antifungal agent.

In one embodiment, where the patient is predicted to be a responder, an antibiotic and/or antifungal agent may be administered.

In a further aspect there is provided a method of treating a patient with reduced liver function, the method comprising the steps of:
a) measuring the concentration of ethanol, one or more sugar fermentation product, such as 2,3-butanediol, and/or a direct ethanol biomarker in a biological sample obtained from the patient; and
b) predicting a response to treatment with an antibiotic and/or antifungal agent by comparing the concentration to a reference value from a subject with reduced liver function that does not respond to therapy with an antibiotic and/or antifungal agent; and/or from a subject with reduced liver function that does respond to therapy with an antibiotic and/or antifungal agent; and/or from a healthy subject;
   wherein if the reference level is from a subject that does not respond to therapy with an antibiotic and/or antifungal agent, then an increase in the concentration of ethanol, one or more sugar fermentation product, such as 2,3-butanediol, and/or a direct ethanol biomarker compared to the reference value is indicative that the patient will respond to therapy with an antibiotic and/or antifungal agent;
   wherein if the reference level is from a subject that does respond to therapy with an antibiotic and/or antifungal agent, then the same or substantially the same or an increase in concentration of ethanol, one or more sugar fermentation product, such as 2,3-butanediol, and/or a direct ethanol biomarker is indicative that the patient will respond to therapy with an antibiotic and/or antifungal agent;
   or wherein if the reference value is from a healthy subject then an increase in the concentration of ethanol, one or more sugar fermentation product, such as 2,3-butanediol, and/or a direct ethanol biomarker compared to the reference value is indicative that the patient will respond to therapy with an antibiotic and/or antifungal agent; and
c) if the patient is predicted to respond to therapy with an antibiotic and/or antifungal agent, administering an antibiotic and/or antifungal agent to the patient.

### FIGURES

The invention is further described in the following non-limiting figures.

FIG. 1: Glucose induced dynamic changes in blood alcohol concentration (BAC) in HiAlc-Kpn-fed (High alcohol producing Klebsiella pneumoniae) mice after 4, 6, and 8 weeks, which were measured at 2, 2.5, 3, 3.5, 4, and 4.5 h after feeding (Jing Yuan et al, Fatty Liver Disease Caused by High-Alcohol Producing Klebsiella pneumoniae, Cell Metabolism 30, 675-688, October 1, 2-19).

FIG. 2: Concentration of ethanol in breath and blood samples taken from a woman suffering from symptoms of ethanol intoxication after a meal made of 78% carbohydrates (H. Kaji et al, Intragastrointestinal Alcohol Fermentation Syndrome: Report of Two Cases and Review of the Literature, Journal of the Forensic Science Society, 24-5, 461-471, September 1984).

FIG. 3: Metabolic fermentation process of glucose

FIG. 4: Levels of ethanol measured in the ambient before and after the procedure, or at baseline before glucose administration.

FIG. 5: Levels of breath ethanol before and after glucose administration.

FIG. 6: Baseline levels of acetoin and 2-3 butanediol.

FIG. 7: Breath profile of acetoin after glucose administration.

### DETAILED DESCRIPTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

In a first aspect there is provided a method for identifying a patient having reduced liver-function or a patient at risk of reduced liver function that will respond to therapy with an antibiotic and/or antifungal agent comprising measuring the concentration of ethanol, one or more sugar fermentation product, such as 2,3-butanediol, and/or a direct ethanol biomarker in a biological sample from said patient, wherein the concentration of ethanol, one or more sugar fermentation product, such as 2,3-butanediol, and/or a direct ethanol biomarker is indicative that said patient will respond to therapy with an antibiotic and/or antifungal agent.

The patient may have reduced liver function or may be at risk of reduced liver function.

The patient may have reduced liver function due to obesity, having type 2 diabetes and/or having a liver disease such as non-alcoholic fatty liver disease (NAFLD) or non-alcoholic steatohepatitis (NASH), cirrhosis, etc. The patient may have reduced liver function as a result of having a liver disease. For example, the patient may have been diagnosed with a liver disease.

The patient may have a liver disease, such as non-alcoholic fatty liver disease (NAFLD) or non-alcoholic steatohepatitis (NASH).

NAFLD is a term that refers to a range of conditions which are caused by a build-up of fat in the liver. The term encompasses a disease spectrum which includes a mild benign form of the disease where there is a build-up of fat in the liver, referred to as steatosis or NAFL. This disease state can progress to a more severe form known as NASH wherein the liver becomes inflamed, this stage may also be referred to herein as NASH without fibrosis. NASH can then progress to NASH with fibrosis, where persistent inflammation causes scar tissue around the liver and nearby blood vessels to form. The most severe form of the disease is NASH with cirrhosis which can occur after long term inflammation, resulting in shrinkage and scarring of the liver. NASH with cirrhosis causes permanent damage to the liver and can lead to liver failure and development of liver cancer (hepatocellular carcinoma (HCC)).

The severity of NAFLD can be described among three stages. Stage 1 is characterized by simple fatty liver (i.e. NAFL or hepatic steatosis). Fat begins to accumulate in individual cells but liver function is normal. There are usually no symptoms and patients may not realize they have the condition. Although the fat deposits are considered harmless, it is important to prevent the disease from progressing to the next stage.

Stage 2 is often referred to as NASH. NASH is a more aggressive form of the condition, where the liver has become inflamed. Inflammation is the body's healing response to damage or injury and, in this case, is a sign that liver cells have become damaged. A person with NASH may have a dull or aching pain felt in the top right of their abdomen (over the lower right side of their ribs). NASH can occur with or without fibrosis.

Stage 3 is often characterized by cirrhosis. At this most severe stage, bands of scar tissue and clumps of liver cells develop. The liver shrinks and becomes lumpy which is known as cirrhosis. Cirrhosis progresses slowly gradually causing the liver to stop functioning. The damage caused by cirrhosis is irreversible and the patient may experience signs of liver failure. Cirrhosis tends to occur after the age of 50, usually after years of liver inflammation associated with the early stages of the disease. People with cirrhosis of the liver caused by NAFLD often also have type 2 diabetes.

The term “cirrhosis,” “liver cirrhosis” or “hepatic cirrhosis” refers to a condition in which the liver does not function properly due to long-term damage. This damage is characterized by the replacement of normal liver tissue by scar tissue (i.e. fibrosis). The disease generally develops slowly over months or years, often with no symptoms. Eventually, excessive scar formation will result in loss of liver function.

The term “prognosis” refers to the forecast or likely outcome of a disease. As used herein, it refers to the probable outcome of liver disease, including whether the disease (e.g. NASH) will respond to treatment or mitigation efforts and/or the likelihood that the disease will progress.

The term progression as used herein may refer to an advancement of the disease state. The term regression as used herein may refer to a decrease of the severity of the disease state. When the disease is monitored, this can result in detecting progression or regression.

Regression may be due to health style changes or therapeutic intervention, for example using a treatment as described herein, including a treatment in clinical drug trials.

The patient may be at risk of reduced liver function. By “at risk of reduced liver function” is meant that a patient has, or has been exposed to, one or more risk factors for liver disease, i.e., one or more factors that are known to increase the likelihood of a subject developing a liver disease. For example, the patient may have one or more risk factors selected from, but not limited to, obesity, alcoholism, family history of liver disease, type 2 diabetes, dyslipidaemia, bowel disease, a genetic predisposition to a liver disease and/or any other disease, syndrome and/or pathology that result in an increased likelihood of gut-related problems. In such embodiments, the patient typically has not previously been diagnosed with a liver disease.

By “alcoholism”, and like terms as used herein, is meant a patient, or subject, that regularly exceeds a recommended intake of alcohol. For example, the patient, or subject, may intake an average of at least 10 grams (g) of alcohol per day. It has been shown that an intake of alcohol of at least 10 g per day increases the risk of hepatic events in subjects with NAFLD.

The method comprises measuring the concentration of ethanol, one or more sugar fermentation product, such as 2,3-butanediol, and/or a direct ethanol biomarker in a biological sample from the patient.

The term “direct ethanol biomarker” is well known in the art and refers to the products of direct ethanol biotransformation/metabolism. Examples of suitable direct ethanol biomarkers include, but are not limited to, ethyl sulphate, ethyl-glucuronide, fatty acid ethyl esters, phosphatidylethanol, and/or combinations thereof.

The term “sugar fermentation product” refers to any compound formed during the metabolism of sugar in bacteria and/or yeast. It will be appreciated that a sugar fermentation product is therefore a compound formed during the metabolism of a sugar in bacteria and/or yeast that may be present in the gut, mouth and/or urinary tract of a patient. The sugar may be glucose, such as 1,5-glucose. As such, a sugar fermentation product may be any compound formed during the metabolism of glucose in bacteria and/or yeast. Examples of suitable sugar fermentation products include, but are not limited to, pyruvate, formic acid and/or ester derivates thereof, acetylphosphate, acetylaldehyde, hydroxyethyl-thiamine pyrophosphate (hydroxyethyl-TPP), acetyllactate, acetoin, 2,3-butanediol and/or combinations thereof. The sugar fermentation product may be 2,3-butanediol.

The method may comprise measuring the concentration of ethanol.

The term “biological sample” may be used interchangeably with the term biological matrix. The biological sample or matrix may be selected from breath, stool, urine, blood, serum, and/or tissue. The biological sample may be a tissue sample such as adipose tissue, liver, brain, bone marrow, muscle or hair. The biological sample may be a sample of bodily fluid. Methods are well known in the art for obtaining bodily fluid samples. The bodily fluid sample may be a sample of blood, stool, urine or exhaled breath. The sample of blood may comprise one or more of blood plasma, red blood cells, white blood cells, platelets. The blood sample may comprise any combination of blood plasma, red blood cells, white blood cells, platelets.

The biological sample may be hair, blood, stool, urine and/or exhaled breath, for example blood, urine and/or exhaled breath, for example blood, urine and/or exhaled breath.

The biological sample may be exhaled breath.

Where the biological sample is a sample of exhaled breath, the breath sample can include air exhaled from one or more different parts of the patient's body (e.g. nostrils, pharynx, trachea, bronchioles, alveoli etc.). For the collection of a breath sample and methods of measurement, the device and methods described in WO2017/187120 or WO2017/187141 (both publications are hereby incorporated by reference) can be used.

In embodiments wherein the biological sample is a sample of exhaled breath, this may be obtained by collecting exhaled air from the patient, for example by requesting the subject to exhale air into a gas-sampling container, such as a bag, a bottle or any other suitable gas-sampling product. Preferably the gas-sampling container resists gas permeation both into and out of the bag and/or is chemically inert, thereby assuring sample integrity. Exhaled breath may also be collected using a breath collector apparatus. Preferably, collection of a sample of exhaled breath is performed in a minimally invasive or a non-invasive manner.

The determination of the amount of ethanol, one or more sugar fermentation product, such as 2,3-butanediol, and/or a direct ethanol biomarker in a sample of exhaled breath from a patient may be performed by the use of at least one technique including, but not limited to, Gas-Chromatography (GC), Gas-Chromatography-lined Mass Spectrometry (GC/MS), Liquid Chromatography-tandem mass spectrometry (LC/MS), Ion Mobility Spectrometry/Mass Spectrometry (IMS/MS), Proton Transfer Reaction Mass-Spectrometry (PTR-MS), Electronic Nose device, quartz crystal microbalance or chemically sensitive sensors.

The amount of ethanol, one or more sugar fermentation product, such as 2,3-butanediol, and/or a direct ethanol biomarker in a sample of exhaled breath from a patient may be determined using thermal desorption-gas chromatography-time of flight-mass spectrometry (GC-tof-MS). In certain embodiments, breath of the patient is collected in an inert bag, then the content of the bag is transported under standardised conditions onto desorption tubes and VOCs are analyzed by thermally desorbing the content of the tube and then separated by capillary gas chromatography. Then volatile organic peaks corresponding to the compound of interest (i.e., ethanol, one or more sugar fermentation product, such as 2,3-butanediol, and/or a direct ethanol biomarker) are detected with MS and identified using for example a library, such as the National Institute of Standards and Technology. Thermal desorption may be performed at the GC inlet at a temperature of, e.g., about 200-350° C. In all chromatography, separation occurs when the sample mixture is introduced (injected) into a mobile phase. Gas chromatography (GC) typically uses an inert gas such as helium as the mobile phase. GC/MS allows for the separation, identification and/or quantification of individual components from a biological sample. MS methods which may be used with the present invention include, but are not limited to, electron ionization, electrospray ionization, glow discharge, field desorption (FD), fast atom bombardment (FAB), thermospray, desorption/ionization on silicon (DIOS), Direct Analysis in Real Time (DART), atmospheric pressure chemical ionization (APCI), secondary ion mass spectrometry (SIMS), spark ionization and thermal ionization (TIMS). Matrix assisted laser desorption ionization time-of-flight mass spectrometry (MALDI-TOF-MS) is an example of a mass spectroscopy method which may be used to determine the amount of ethanol, one or more sugar fermentation product, such as 2,3-butanediol, and/or a direct ethanol biomarker from a sample of exhaled breath from a patient.

The method may comprise collecting different selected exhaled breath samples, or fractions thereof, on a single breath sample capture device. For example, the method may comprise the steps of:
(a) collecting a first exhaled breath sample by contacting the sample with a capture device comprising an adsorbent material;
(b) collecting a second exhaled breath sample by contacting the second sample with said capture device, wherein the first and second exhaled breath samples are caused to be captured on the capture device in a spatially separated manner.

The capture device may comprise an adsorbent material in the form of a porous polymeric resin. Suitable adsorbent materials include Tenax® resins and Carbograph® materials. Tenax® is a porous polymeric resin based on a 2,6-diphenyl-p-propylene oxide monomer. Carbograph® materials are graphitized carbon blacks. In one embodiment, the material is Tenax GR, which comprises a mixture of Tenax® TA and 30% graphite. One Carbograph® adsorbent is Carbograph 5TD. The capture device may comprise both Tenax GR and Carbograph 5TD. The capture device is conveniently a sorbent tube. These are hollow metal cylinders, typically of standard dimensions (3½ inches in length with a ¼ inch internal diameter) packed with a suitable adsorbent material.

The concentration of ethanol, one or more sugar fermentation product, such as 2,3-butanediol, and/or a direct ethanol biomarker measured in the biological sample from a patient is indicative that said patient will respond to therapy with an antibiotic and/or antifungal agent.

When the biological sample is blood, the concentration of ethanol, one or more sugar fermentation product, such as 2,3-butanediol, and/or a direct ethanol biomarker that is indicative that a patient will respond to therapy with an antibiotic and/or antifungal agent may be at least 1, such as 2, 3, 4, 5, 6, 7, 8, 9, 10, 11, 12, 13, 14, 15, 16, 17, 18, 19 or 20 mmol/L.

When the biological sample is exhaled breath, the concentration of ethanol, one or more sugar fermentation product, such as 2,3-butanediol, and/or a direct ethanol biomarker that is indicative that a patient will respond to therapy with an antibiotic and/or antifungal agent may be at least 0.0001, such as 0.0002, 0.0003, 0.0004, 0.0005. 0.0006, 0.0007, 0.0008, 0.0009, 0.001, 0.002, 0.003, 0.004, 0.005, 0.006, 0.007, 0.008, 0.009 or 0.01 mmol/L.

The likelihood of a response to therapy with an antibiotic and/or antifungal agent may be determined by comparing the concentration of ethanol, one or more sugar fermentation product, such as 2,3-butanediol, and/or a direct ethanol biomarker in a biological sample obtained from the patient to one or more reference values.

As such, the method may involve measuring the concentration of ethanol, one or more sugar fermentation product, such as 2,3-butanediol, and/or a direct ethanol biomarker in a biological sample obtained from the patient and comparing the concentration to a reference value.

As used herein, “reference value”, “reference values” etc. means the concentration, or concentrations, of ethanol, one or more sugar fermentation product, such as 2,3-butanediol, and/or a direct ethanol biomarker determined by performing the method on one or more reference subjects. “Reference values” are used interchangeably with “reference concentrations” herein. A “test value”, “test concentration”, etc., as used herein, refers to the concentration, or concentrations, of ethanol, one or more sugar fermentation product, such as 2,3-butanediol, and/or a direct ethanol biomarker determined by performing the method on the patient. As such, the test value is suitably the concentration of ethanol, one or more sugar fermentation product, such as 2,3-butanediol, and/or a direct ethanol biomarker in a biological sample obtained from the patient and the reference value is suitably the concentration of ethanol, one or more sugar fermentation product, such as 2,3-butanediol, and/or a direct ethanol biomarker in a biological sample obtained from a reference subject.

As discussed herein, subjects that respond to therapy with an antibiotic and/or antifungal agent may have an overgrowth of bacteria and/or yeast in the gut, mouth and/or urinary tract compared to those that do not respond to therapy with an antibiotic and/or antifungal agent. For example, the bacteria and/or yeast that is present in the gut, mouth and/or urinary tract of a subject that responds to therapy with an antibiotic and/or antifungal agent may have an abundance of ethanol producing bacteria and/or yeast. In contrast, the bacteria and/or yeast present in the gut, mouth and/or urinary tract of a subject that does not respond to therapy with an antibiotic and/or antifungal agent may have low, or reduced, levels of ethanol-producing bacteria and/or yeast compared to a subject that does respond to therapy with an antibiotic and/or antifungal agent.

The reference subject may be a subject that does not respond to therapy with an antibiotic and/or antifungal agent. A subject that does not respond to therapy with an antibiotic and/or antifungal agent will typically produce lower levels of ethanol, one or more sugar fermentation product, such as 2,3-butanediol, and/or a direct ethanol biomarker compared to subjects that do respond to therapy with an antibiotic and/or antifungal agent. In such embodiments, an increase in the concentration of ethanol, one or more sugar fermentation product, such as 2,3-butanediol, and/or a direct ethanol biomarker in a biological sample obtained from the patient (i.e., the “test value”) compared to the reference value is indicative that the patient will respond to therapy with an antibiotic and/or antifungal agent.

The reference subject may be a subject that does respond to therapy with an antibiotic and/or antifungal agent. A subject that responds to therapy with an antibiotic and/or antifungal agent will typically produce higher levels of ethanol, one or more sugar fermentation product, such as 2,3-butanediol, and/or a direct ethanol biomarker compared to subjects that do not respond to therapy with an antibiotic and/or antifungal agent. In such embodiments, the same or substantially the same or an increase in concentration of ethanol, one or more sugar fermentation product, such as 2,3-butanediol, and/or a direct ethanol biomarker in a biological sample obtained from the patient (i.e., the “test value”) compared to the reference value is indicative that the patient will respond to therapy with an antibiotic and/or antifungal agent.

The reference subject may be a healthy subject. A healthy subject will typically produce lower levels of ethanol, one or more sugar fermentation product, such as 2,3-butanediol, and/or a direct ethanol biomarker compared to patients having reduced liver-function or at risk of reduced liver function. In such embodiments, an increase in the concentration of ethanol, one or more sugar fermentation product, such as 2,3-butanediol, and/or a direct ethanol biomarker in a biological sample obtained from the patient (i.e., the “test value”) compared to the reference value is indicative that the patient will respond to therapy with an antibiotic and/or antifungal agent.

The method may comprise the steps of:
a) measuring the concentration of ethanol, one or more sugar fermentation product, such as 2,3-butanediol, and/or a direct ethanol biomarker in a biological sample obtained from the patient; and
b) comparing the concentration to a reference value from a subject with reduced liver function that does not respond to therapy with an antibiotic and/or antifungal agent; and/or from a subject with reduced liver function that does respond to therapy with an antibiotic and/or antifungal agent; and/or from a healthy subject;
   wherein if the reference value is from a subject that does not respond to therapy with an antibiotic and/or antifungal agent, then an increase in the concentration of ethanol, one or more sugar fermentation product, such as 2,3-butanediol, and/or a direct ethanol biomarker compared to the reference value is indicative that the patient will respond to therapy with an antibiotic and/or antifungal agent;
   wherein if the reference value is from a subject that does respond to therapy with an antibiotic and/or antifungal agent, then the same or substantially the same or an increase in concentration of ethanol, one or more sugar fermentation product, such as 2,3-butanediol, and/or a direct ethanol biomarker compared to the reference value is indicative that the subject will respond to therapy with an antibiotic and/or antifungal agent;
   or wherein if the reference value is from a healthy subject then an increase in the concentration of ethanol, one or more sugar fermentation product, such as 2,3-butanediol, and/or a direct ethanol biomarker compared to the reference value is indicative that the patient will respond to therapy with an antibiotic and/or antifungal agent.

The concentration of ethanol, one or more sugar fermentation product, such as 2,3-butanediol, and/or a direct ethanol biomarker in a biological sample obtained from the patient may be compared to one or more reference value. Reference to “a” reference value as herein includes a single reference value, i.e., the concentration of ethanol, one or more sugar fermentation product, such as 2,3-butanediol, and/or a direct ethanol biomarker in a biological sample taken from a reference subject, and multiple, such as 2, 3, 4, 5, 10, 50, 100, etc., reference values, i.e., the concentration of ethanol, one or more sugar fermentation product, such as 2,3-butanediol, and/or a direct ethanol biomarker in biological samples taken from two or more reference subjects.

In one embodiment, the reference value may be an average of reference values taken from multiple, such as 2, 3, 4, 5, 10, 50, 100, etc., reference subjects. For example, the reference value may be the average concentration of ethanol, one or more sugar fermentation product, such as 2,3-butanediol, and/or a direct ethanol biomarker in biological samples taken from multiple, such as 2, 3, 4, 5, 10, 50, 100, etc., subjects that do not respond to therapy with an antibiotic and/or antifungal agent. For example, the reference value may be the average concentration of ethanol, one or more sugar fermentation product, such as 2,3-butanediol, and/or a direct ethanol biomarker in biological samples taken from multiple, such as 2, 3, 4, 5, 10, 50, 100, etc., subjects that do not respond to therapy with an antibiotic and/or antifungal agent.

The concentration of ethanol, one or more sugar fermentation product, such as 2,3-butanediol, and/or a direct ethanol biomarker in a biological sample obtained from the patient may be compared to a reference value from a subject with reduced liver function that does not respond to therapy with an antibiotic and/or antifungal agent.

The concentration of ethanol, one or more sugar fermentation product, such as 2,3-butanediol, and/or a direct ethanol biomarker in a biological sample obtained from the patient may be compared to a reference value from a subject with reduced liver function that does respond to therapy with an antibiotic and/or antifungal agent.

The concentration of ethanol, one or more sugar fermentation product, such as 2,3-butanediol, and/or a direct ethanol biomarker in a biological sample obtained from the patient may be compared to a reference value from a subject with reduced liver function that does not respond to therapy with an antibiotic and/or antifungal agent and from a subject with reduced liver function that does respond to therapy with an antibiotic and/or antifungal agent. When the concentration of ethanol, one or more sugar fermentation product, such as 2,3-butanediol, and/or a direct ethanol biomarker in a biological sample obtained from the patient may be compared to a reference value from a subject with reduced liver function that does not respond to therapy with an antibiotic and/or antifungal agent and from a subject with reduced liver function that does respond to therapy with an antibiotic and/or antifungal agent, the methods of the invention may be more accurate.

When the biological sample is blood and the reference value is from a subject that does not respond to therapy with an antibiotic and/or antifungal agent, the concentration of ethanol, one or more sugar fermentation product, such as 2,3-butanediol, and/or a direct ethanol biomarker in a biological sample taken from the patient may be at least 0.1, such as 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19 or 20 mmol/L higher than the reference value.

When the biological sample is exhaled breath and the reference value is from a subject that does not respond to therapy with an antibiotic and/or antifungal agent, the concentration of ethanol, one or more sugar fermentation product, such as 2,3-butanediol, and/or a direct ethanol biomarker in a biological sample taken from the patient may be at least 0.0001, such as 0.0002, 0.0003, 0.0004, 0.0005. 0.0006, 0.0007, 0.0008, 0.0009, 0.001, 0.002, 0.003, 0.004, 0.005, 0.006, 0.007, 0.008, 0.009 or 0.01 mmol/L higher than the reference value.

When the biological sample is blood and the reference value is from a subject that does respond to therapy with an antibiotic and/or antifungal agent, the concentration of ethanol, one or more sugar fermentation product, such as 2,3-butanediol, and/or a direct ethanol biomarker in a biological sample taken from the patient may be the same, substantially the same or at least 0.1, such as 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19 or 20 mmol/L higher than the reference value.

When the biological sample is exhaled breath and the reference value is from a subject that does respond to therapy with an antibiotic and/or antifungal agent, the concentration of ethanol, one or more sugar fermentation product, such as 2,3-butanediol, and/or a direct ethanol biomarker in a biological sample taken from the patient may be the same, substantially the same or at least 0.0001, such as 0.0002, 0.0003, 0.0004, 0.0005. 0.0006, 0.0007, 0.0008, 0.0009, 0.001, 0.002, 0.003, 0.004, 0.005, 0.006, 0.007, 0.008, 0.009 or 0.01 mmol/L higher than the reference value.

When the biological sample is blood and the reference value is from a healthy subject, the concentration of ethanol, one or more sugar fermentation product, such as 2,3-butanediol, and/or a direct ethanol biomarker in a biological sample taken from the patient may be at least 0.1, such as 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19 or 20 mmol/L higher than the reference value.

When the biological sample is exhaled breath and the reference value is from a healthy subject, the concentration of ethanol, one or more sugar fermentation product, such as 2,3-butanediol, and/or a direct ethanol biomarker in a biological sample taken from the patient may be at least 0.0001, such as 0.0002, 0.0003, 0.0004, 0.0005. 0.0006, 0.0007, 0.0008, 0.0009, 0.001, 0.002, 0.003, 0.004, 0.005, 0.006, 0.007, 0.008, 0.009 or 0.01 mmol/L higher than the reference value.

It will be appreciated by a person skilled in the art that ethanol, one or more sugar fermentation product, such as 2,3-butanediol, and/or a direct ethanol biomarker are each volatile organic compounds (VOCs) that may be secreted in biological samples. Generally, VOCs are defined as organic chemical compounds whose composition makes it possible for them to evaporate under normal indoor atmospheric conditions of temperature and pressure. Since the volatility of a compound is generally higher the lower its boiling point temperature, the volatility of organic compounds is sometimes defined and classified by their boiling points. Volatile compounds are compounds that are secreted by the human body into gas fluids, including for example breath, skin emanations and others. As such, the ethanol, one or more sugar fermentation product, such as 2,3-butanediol, and/or a direct ethanol biomarker may be measured in a biological sample, such as breath, without the use of any labels, such as isotope labels.

The method may further comprise administering a carbohydrate to the patient. Preferably, the step of administering a carbohydrate to the patient is carried out prior to the step of obtaining a test sample from the patient. Preferably, the step of administering a carbohydrate to the patient is carried out after the step of obtaining a reference sample from the patient. For the avoidance of doubt, a “reference sample” in this context means a sample taken from a patient before the administration of a carbohydrate for the purposes of determining whether, or not, the administration of the carbohydrate results in an increased level of ethanol. Advantageously, the administration of a carbohydrate to a patient prior to obtaining a test sample from the patient may increase the sensitivity of the method. Advantageously, the administration of a carbohydrate to a patient after the step of obtaining a reference sample from the patient may increase the sensitivity of the method and/or may make the test more informative. For example, it can be demonstrated whether or not the administration of a carbohydrate induces an increase in ethanol, one or more sugar fermentation product, such as 2,3-butanediol, and/or direct ethanol biomarker levels.

The carbohydrate may be any suitable carbohydrate. The carbohydrate may be provided in any suitable form. For example, the carbohydrate may be provided in the form of a tablet, capsule, a foodstuff, a liquid, such as a beverage, and/or any other oral administration format.

The patient may have fasted before the step of administering a carbohydrate and/or sugar. For example, the patient may have been fasting overnight before the step of administering a carbohydrate and/or sugar.

The carbohydrate may be labelled or unlabelled.

The carbohydrate may be labelled such that at least a portion of the ethanol, one or more sugar fermentation product, such as 2,3-butanediol, and/or direct ethanol biomarker detected is labelled.

Preferably, the method may comprise measuring the concentration of ethanol in a biological sample obtained from the patient and at least a portion of the ethanol detected may be labelled. The label may be 12C, 13C, 14C, 2H, 17O and/or 18O, such as 13C.

Thus, the methods may use ethanol, one or more sugar fermentation product, such as 2,3-butanediol, and/or direct ethanol biomarker as an exogenous volatile organic compound (EVOC), wherein the EVOC is a tracer of specific in vivo liver-specific metabolic activities. EVOCs can be volatile compounds that, administered to a subject through various routes, undergo metabolism and distribution in the body and are excreted via breath. Additionally, when the exogenous compound is a substrate for an enzyme, metabolism of EVOCs by liver-specific enzymes can lead to production of other volatile compounds that can also be detected in breath.

It will be appreciated by the skilled person that for this application the carbohydrate is an exogenous compound. An “exogenous compound” is any compound that can be administered to a patient that is metabolised within the patient (i.e., to form ethanol). An exogenous compound refers to a chemical compound that is recognized by the gut microbiome, such as by one or more enzyme(s) in the gut microbiome, and which is converted into a different chemical compound which is referred to herein as a “metabolite”. An “exogenous compound” is a xenobiotic, i.e., a substance that is foreign to the patient's body and which is specifically and selectively metabolised in vivo.

In one embodiment, the reference value may be the concentration of ethanol, one or more sugar fermentation product, such as 2,3-butanediol, and/or a direct ethanol biomarker in a biological sample obtained from the patient before a carbohydrate has been administered. For example, the reference value may be the concentration of ethanol, one or more sugar fermentation product, such as 2,3-butanediol, and/or a direct ethanol biomarker in a biological sample taken from a patient who has fasted for at least 1 hour, such as at least 2, 3, 4, 5, 6, 7 or 8 hours. The reference value may be the concentration of ethanol, one or more sugar fermentation product, such as 2,3-butanediol, and/or a direct ethanol biomarker in a biological sample taken from a patient who has fasted overnight. In such embodiments, the “test value” will suitably be the concentration of ethanol, one or more sugar fermentation product, such as 2,3-butanediol, and/or a direct ethanol biomarker in a biological sample taken from the patient after a carbohydrate has been administered. For example, the test value may be the concentration of ethanol, one or more sugar fermentation product, such as 2,3-butanediol, and/or a direct ethanol biomarker in a biological sample taken from the patient up to 1, 2, 3, 4 etc. hours after a carbohydrate has been administered. In such embodiments, the difference in the concentration of ethanol, one or more sugar fermentation product, such as 2,3-butanediol, and/or a direct ethanol biomarker in the test value compared to the concentration of ethanol, one or more sugar fermentation product, such as 2,3-butanediol, and/or a direct ethanol biomarker in the reference value may be indicative that said patient will respond to therapy with an antibiotic and/or antifungal agent if said difference is above a threshold value.

Thus, the method may comprise the steps of:
a) establishing a reference value by measuring the concentration of ethanol, one or more sugar fermentation product, such as 2,3-butanediol, and/or a direct ethanol biomarker in a biological sample obtained from the patient before a carbohydrate has been administered;
b) administering a carbohydrate to the patient;
c) establishing a test value by measuring the concentration of ethanol, one or more sugar fermentation product, such as 2,3-butanediol, and/or a direct ethanol biomarker in a biological sample obtained from the patient after the carbohydrate has been administered; and
d) comparing the reference value to the test value, wherein the reference value is the concentration of ethanol, one or more sugar fermentation product, such as 2,3-butanediol, and/or a direct ethanol biomarker in a biological sample obtained from the patient before a carbohydrate has been administered;
   wherein the difference in the concentration of ethanol, one or more sugar fermentation product, such as 2,3-butanediol, and/or a direct ethanol biomarker in the test value compared to the concentration of ethanol, one or more sugar fermentation product, such as 2,3-butanediol, and/or a direct ethanol biomarker in the reference value is indicative that said patient will respond to therapy with an antibiotic and/or antifungal agent if said difference is above a threshold value.

The threshold value may be any suitable value.

For example, when the biological sample is blood the threshold value, i.e., the difference between the concentration of ethanol, one or more sugar fermentation product, such as 2,3-butanediol, and/or a direct ethanol biomarker in the test value compared to the concentration of ethanol, one or more sugar fermentation product, such as 2,3-butanediol, and/or a direct ethanol biomarker in the reference value, may be at least 0.1, such as 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20 mmol/L, or higher.

For example, when the biological sample is exhaled breath the threshold value, i.e., the difference between the concentration of ethanol, one or more sugar fermentation product, such as 2,3-butanediol, and/or a direct ethanol biomarkers in the test value compared to the concentration of ethanol, one or more sugar fermentation product, such as 2,3-butanediol, and/or a direct ethanol biomarker in the reference value, may be at least 0.0001, such as 0.0002, 0.0003, 0.0004, 0.0005. 0.0006, 0.0007, 0.0008, 0.0009, 0.001, 0.002, 0.003, 0.004, 0.005, 0.006, 0.007, 0.008, 0.009, 0.01 mmol/L or higher.

The step of administering a carbohydrate to the patient may be carried out at any suitable time point. For example, the step of administering a carbohydrate to the patient may be carried out at least 10, such as 20, 30, 40, 50, 60, 90, 120, or 180 minutes prior to the step of obtaining a biological sample from the patient.

The method may further comprise the step of administering an alcohol dehydrogenase (ADH) inhibitor to the patient. Preferably, the step of administering an ADH inhibitor to the patient is carried out prior to the step of obtaining a biological sample from the patient.

Alcohol metabolism is a well-characterized biological process that is dominated by the alcohol dehydrogenase (ADH) and aldehyde dehydrogenase (ALDH) families. Alcohol dehydrogenases catalyse the oxidation of primary and secondary alcohols to the corresponding aldehyde or ketone. Alterations in alcohol metabolism processes in response to human NASH progression have been investigated and the activity and expression of Alcohol dehydrogenase enzymes has been studied (Li, H., Toth, E. & Cherrington, N. J. Alcohol Metabolism in the Progression of Human Nonalcoholic Steatohepatitis. Toxicol Sci 164, 428-438, (2018)). As such, administering an alcohol dehydrogenase (ADH) inhibitor to the patient may advantageously increase the amount of ethanol and/or a direct ethanol biomarker that can be detected. For example, administering an alcohol dehydrogenase (ADH) inhibitor may advantageously reduce the amount of ethanol and/or a direct ethanol biomarker that is metabolised in vivo.

The ADH inhibitor may be an aa-alcohol dehydrogenase inhibitor. The ADH inhibitor may be ALDH4A1, ADH1A, ADH1B, and/or ADH4 inhibitor.

The ADH inhibitor may be 4-methylpyrazole

The step of administering an ADH inhibitor to the patient, may be carried out at any suitable time point. For example, the step of administering an alcohol dehydrogenase (ADH) inhibitor to the patient may be carried out prior to the step of obtaining a biological sample from the patient. For example, the step of administering an ADH inhibitor to the patient may be carried out least 10, such as 20, 30, 40, 50, 60, 90, 120, or 180 minutes prior to the step of obtaining a biological sample from the patient.

In embodiments where a carbohydrate is administered to a patient, the step of administering an ADH inhibitor to the patient may be carried out before, at the same time as and/or after the step of administering a carbohydrate to a patient. Preferably, the step of administering an ADH inhibitor to the patient may be carried before or at the same time as the step of administering a carbohydrate to a patient, for example before the step of administering a carbohydrate to a patient.

The biological sample may be obtained at any suitable time point.

The biological sample may suitably be obtained before therapy with an antibiotic and/or antifungal agent.

In one embodiment, the biological sample be obtained after therapy with an antibiotic and/or antifungal agent, for example, to determine the efficacy of said therapy.

In one embodiment, the biological sample may be obtained before and after therapy with an antibiotic and/or antifungal agent, for example, to determine the efficacy of said therapy.

The method may comprise the step of identifying a patient in need of treatment with an antibiotic and/or antifungal agent.

The method may comprise administering an antibiotic and/or antifungal agent to the patient.

Suitably, the step of administering an antibiotic and/or antifungal agent to the patient is carried out only when said patient has been identified as likely to respond to treatment with an antibiotic and/or antifungal agent.

The antibiotic may be any suitable antibiotic. The antibiotic may comprise rifaximin; neomycin; metronidazole; flouroquinolones, such as norfloxacin and ciprofloxacin; cephalosporins, such as ceftriaxone and cefotaxime; trimethoprim-sulfamethoxazole; flucloxacillin; cephalothin; aztreonam; cabimicina; and/or combinations thereof.

The antifungal agent may be any suitable antifungal agent. The antifungal agent may comprise clotrimazole, econazole, miconazole, terbinafine, fluconazole, ketoconazole, nystatin, amphotericin, and/or combinations thereof.

The invention also extends to a method of predicting a response to an antibiotic and/or antifungal agent therapy in a patient having reduced liver-function or at risk of reduced liver function comprising measuring the concentration of ethanol, one or more sugar fermentation product, such as 2,3-butanediol, and/or a direct ethanol biomarker in a biological sample from said patient, wherein the concentration of ethanol, one or more sugar fermentation product, such as 2,3-butanediol, and/or a direct ethanol biomarker is indicative of a response or non-response of a patient to therapy with an antibiotic and/or antifungal agent.

Suitable features of this aspect of the invention are as defined herein in relation to the one or more of the other aspects of the invention (as appropriate).

Such methods may comprise the steps of:
a) measuring the concentration of ethanol, one or more sugar fermentation product, such as 2,3-butanediol, and/or a direct ethanol biomarker in a biological sample obtained from the patient; and
b) comparing the concentration to a reference value from a subject with reduced liver function that does not respond to therapy with an antibiotic and/or antifungal agent; and/or from a subject with reduced liver function that does respond to therapy with an antibiotic and/or antifungal agent; and/or from a healthy subject;
   wherein if the reference level is from a subject that does not respond to therapy with an antibiotic and/or antifungal agent, then an increase in the concentration of ethanol, one or more sugar fermentation product, such as 2,3-butanediol, and/or a direct ethanol biomarker compared to the reference value is indicative that the patient will respond to therapy with an antibiotic and/or antifungal agent; or
   wherein if the reference level is from a subject that does respond to therapy with an antibiotic and/or antifungal agent, then the same or substantially the same or an increase in concentration of ethanol, one or more sugar fermentation product, such as 2,3-butanediol, and/or a direct ethanol biomarker is indicative that the patient will respond to therapy with an antibiotic and/or antifungal agent;
   wherein if the reference level is from a healthy subject, then an increase in the concentration of ethanol, one or more sugar fermentation product, such as 2,3-butanediol, and/or a direct ethanol biomarker compared to the reference value is indicative that the patient will respond to therapy with an antibiotic and/or antifungal agent.

The methods may comprise the step of predicting a response. The step of predicting a response may be done by comparing the concentration of ethanol, one or more sugar fermentation product, such as 2,3-butanediol, and/or a direct ethanol biomarker in a biological sample obtained from the patient to one or more reference values (as defined herein).

If the patient is predicted to be a responder, the methods may include the step of administering an antibiotic and/or antifungal agent to the patient. It will be appreciated by a person skilled in the art that where the patient is not predicted to be a responder, i.e., is predicted to be a non-responder, the methods may suitably not include the step of administering an antibiotic and/or antifungal agent.

The methods of the invention may be used in a method of treating a patient with reduced liver function. For example, the methods of the invention may be used in a method of treating a patient with a liver disease. For example, the methods of the invention may be used in a method of treating a liver disease.

Thus, in a further aspect there is provided a method of treating a patient with reduced liver function, the method comprising the steps of:
a) measuring the concentration of ethanol, one or more sugar fermentation product, such as 2,3-butanediol, and/or a direct ethanol biomarker in a biological sample obtained from the patient; and
b) comparing the concentration to a reference value from a subject with reduced liver function that does not respond to therapy with an antibiotic and/or antifungal agent; and/or from a patient with reduced liver function that does respond to therapy with an antibiotic and/or antifungal agent; and/or from a healthy subject;
   wherein if the reference level is from a subject that does not respond to therapy with an antibiotic and/or antifungal agent, then an increase in the concentration of ethanol, one or more sugar fermentation product, such as 2,3-butanediol, and/or a direct ethanol biomarker compared to the reference value is indicative that the patient will respond to therapy with an antibiotic and/or antifungal agent;
   wherein if the reference level is from a subject that does respond to therapy with an antibiotic and/or antifungal agent, then the same or substantially the same or an increase in concentration of ethanol, one or more sugar fermentation product, such as 2,3-butanediol, and/or a direct ethanol biomarker is indicative that the patient will respond to therapy with an antibiotic and/or antifungal agent;
   or wherein if the reference level is from a healthy subject, then an increase in the concentration of ethanol, one or more sugar fermentation product, such as 2,3-butanediol, and/or a direct ethanol biomarker compared to the reference value is indicative that the patient will respond to therapy with an antibiotic and/or antifungal agent; and
c) if the patient is predicted to respond to therapy with an antibiotic and/or antifungal agent, administering an antibiotic and/or antifungal agent to the patient.

Suitable features of this aspect of the invention are as defined herein in relation to the one or more of the other aspects of the invention (as appropriate).

It will be appreciated by a person skilled in the art that where the patient is not predicted to be a responder, i.e., is predicted to be a non-responder, the method of this aspect of the invention may suitably not include the step of administering an antibiotic and/or antifungal agent.

Advantageously, the use of the methods of the present invention mean that an antibiotic and/or antifungal agent treatment may be administered to patients that will benefit from such a treatment. Further, the use of the methods of the invention mean that an antibiotic and/or antifungal agent treatment is only administered to patients that will benefit from such a treatment.

As used herein, unless otherwise expressly specified, all numbers such as those expressing values, ranges, amounts or percentages may be read as if prefaced by the word “about”, even if the term does not expressly appear. Also, the recitation of numerical ranges by endpoints includes all integer numbers and, where appropriate, fractions subsumed within that range (e.g. 1 to 5 can include 1, 2, 3, 4 when referring to, for example, a number of elements, and can also include 1.5, 2, 2.75 and 3.80, when referring to, for example, measurements). The recitation of end points also includes the end point values themselves (e.g. from 1.0 to 5.0 includes both 1.0 and 5.0). Any numerical range recited herein is intended to include all sub-ranges subsumed therein.

The terms “comprising”, “comprises” and “comprised of” as used herein are synonymous with “including”, “includes” or “containing”, “contains”, and are inclusive or open-ended and do not exclude additional, non-recited members, elements or method steps.

As used herein, the term “and/or,” when used in a list of two or more items, means that any one of the listed items can be employed by itself or any combination of two or more of the listed items can be employed. For example, if a list is described as comprising group A, B, and/or C, the list can comprise A alone; B alone; C alone; A and B in combination; A and C in combination, B and C in combination; or A, B, and C in combination.

The invention is further defined in the following numbered aspects:

1. A method for identifying a patient having reduced liver function or a patient at risk of reduced liver function that will respond to therapy with an antibiotic and/or antifungal agent comprising measuring the concentration of ethanol, one or more sugar fermentation product, such as 2,3-butanediol, and/or a direct ethanol biomarker in a biological sample from said patient, wherein the concentration of ethanol, one or more sugar fermentation product, such as 2,3-butanediol, and/or a direct ethanol biomarker is indicative that said patient will respond to therapy with an antibiotic and/or antifungal agent.

2. The method according to aspect 1, comprising the steps of:
a) measuring the concentration of ethanol, one or more sugar fermentation product, such as 2,3-butanediol, and/or a direct ethanol biomarker in a biological sample obtained from the patient; and
b) comparing the concentration to a reference value from a subject with reduced liver function that does not respond to therapy with an antibiotic and/or antifungal agent; and/or from a patient with reduced liver function that does respond to therapy with an antibiotic and/or antifungal agent; and/or a healthy subject;
   wherein if the reference level is from a subject that does not respond to therapy with an antibiotic and/or antifungal agent, then an increase in the concentration of ethanol, one or more sugar fermentation product, such as 2,3-butanediol, and/or a direct ethanol biomarker compared to the reference value is indicative that the patient will respond to therapy with an antibiotic and/or antifungal agent;
   wherein if the reference level is from a subject that does respond to therapy with an antibiotic and/or antifungal agent, then the same or substantially the same or an increase in concentration of ethanol, one or more sugar fermentation product, such as 2,3-butanediol, and/or a direct ethanol biomarker is indicative that the patient will respond to therapy with an antibiotic and/or antifungal agent; or
   wherein if the reference level is from a healthy subject, then an increase in the concentration of ethanol, one or more sugar fermentation product, such as 2,3-butanediol, and/or a direct ethanol biomarker compared to the reference value is indicative that the patient will respond to therapy with an antibiotic and/or antifungal agent.

3. The method according to any one of aspects 1 or 2, wherein the antibiotic and/or antifungal agent is an antibiotic.

4. The method according to any one of aspects 1-3, wherein the method comprises measuring the concentration of ethanol in a biological sample from said patient.

5. The method according to any one of the preceding aspects, wherein the patient has been diagnosed with a liver disease, for example wherein the patient has non-alcoholic fatty liver disease (NAFLD), such as non-alcoholic steatohepatitis (NASH).

6. The method according to any one of aspects 1-4, wherein the patient has not been diagnosed with a liver disease; and wherein the patient is at risk of reduced liver function, for example wherein the patient has one or more risk factors selected from obesity, alcoholism, family history of liver disease, type 2 diabetes, dyslipidemia, and/or a genetic predisposition to a liver disease.

7. The method according to any one of the preceding aspects, further comprising the step of identifying a patient in need of treatment with an antibiotic and/or antifungal agent.

8. The method according to any one of the preceding aspects, further comprising administering an antibiotic and/or antifungal agent to said patient.

9. The method according to any one of the preceding aspects, further comprising the step of obtaining a biological sample from the patient.

10. The method according to any one of the preceding aspects, wherein the biological sample is hair, blood, stool, urine and/or exhaled breath, for example blood, urine and/or exhaled breath, for example wherein the biological sample is exhaled breath.

11. The method according to any one of aspects 9 or 10, further comprising administering a carbohydrate to the patient; wherein the step of administering a carbohydrate to the patient is carried out prior to the step of obtaining a biological sample from the patient.

12. The method according to aspect 11, wherein the carbohydrate is labelled such that at least a portion of the ethanol, one or more sugar fermentation product, such as 2,3-butanediol, and/or a direct ethanol biomarker is labelled.

13. The method according to aspect 12, wherein the presence of labelled ethanol, one or more sugar fermentation product, such as 2,3-butanediol, and/or a direct ethanol biomarker in the biological sample from the patient is indicative that said patient will respond to therapy with an antibiotic and/or antifungal agent.

14. The method according to any one of aspects 12 or 13, wherein the label is 12C, 13C, 14C, 2H, 17O and/or 18O, such as 13C.

15. The method according to any one of aspects 11-14, wherein the step of administering a carbohydrate to the patient is carried out at least 10, such as 20, 30, 40, 50, 60, 90, 120 or 180 minutes prior to the step of obtaining a biological sample from the patient.

16. The method according to any one of aspects 9-15, further comprising the step of administering an alcohol dehydrogenase (ADH) inhibitor to the patient; wherein the step of administering an ADH inhibitor to the patient is carried out prior to the step of obtaining a biological sample from the patient.

17. The method according to aspect 16, wherein, where the method includes the step of administering a carbohydrate to a patient, the step of administering an ADH inhibitor to the patient is carried out before and/or after the step of administering a carbohydrate to a patient.

18. The method according to any one of the preceding aspects, wherein the biological sample is blood; and a concentration of ethanol, one or more sugar fermentation product, such as 2,3-butanediol, and/or a direct ethanol biomarker of at least 20 mmol/L is indicative that said patient will respond to therapy with an antibiotic and/or antifungal agent;
or wherein the biological sample is exhaled breath; and a concentration of ethanol, one or more sugar fermentation product, such as 2,3-butanediol, and/or a direct ethanol biomarker of at least 0.0025 mmol/L, such as at least 0.005 mmol/L, such as at least 0.01 mmol/L, is indicative that said patient will respond to therapy with an antibiotic and/or antifungal agent.

19. The method according to any one of the preceding aspects, wherein the antibiotic comprises rifaximin; neomycin; metronidazole; flouroquinolones, such as norfloxacin and ciprofloxacin; cephalosporins, such as ceftriaxone and cefotaxime; trimethoprim-sulfamethoxazole; flucloxacillin; cephalothin; aztreonam; cabimicina and combinations thereof; and/or wherein the antifungal agent comprises clotrimazole, econazole, miconazole, terbinafine, fluconazole, ketoconazole, nystatin, amphotericin, and/or combinations thereof.

20. A method of predicting a response to an antibiotic and/or antifungal agent therapy in a patient having reduced liver-function or at risk of reduced liver function comprising measuring the concentration of ethanol, one or more sugar fermentation product, such as 2,3-butanediol, and/or a direct ethanol biomarker in a biological sample from said patient, wherein the concentration of ethanol, one or more sugar fermentation product, such as 2,3-butanediol, and/or a direct ethanol biomarker is indicative of a response or non-response of a patient to therapy with an antibiotic and/or antifungal agent.

21. The method according to aspect 20, wherein the method comprises the steps of:
a) measuring the concentration of ethanol, one or more sugar fermentation product, such as 2,3-butanediol, and/or a direct ethanol biomarker in a biological sample obtained from the patient; and
b) comparing the concentration to a reference value from a subject with reduced liver function that does not respond to therapy with an antibiotic and/or antifungal agent; and/or from a subject with reduced liver function that does respond to therapy with an antibiotic and/or antifungal agent; and/or from a healthy subject;
   wherein if the reference level is from a subject that does not respond to therapy with an antibiotic and/or antifungal agent, then an increase in the concentration of ethanol, one or more sugar fermentation product, such as 2,3-butanediol, and/or a direct ethanol biomarker compared to the reference value is indicative that the patient will respond to therapy with an antibiotic and/or antifungal agent;
   wherein if the reference level is from a subject that does respond to therapy with an antibiotic and/or antifungal agent, then the same or substantially the same or an increase in concentration of ethanol, one or more sugar fermentation product, such as 2,3-butanediol, and/or a direct ethanol biomarker is indicative that the patient will respond to therapy with an antibiotic and/or antifungal agent; or
   wherein if the reference level is from a healthy subject, then an increase in the concentration of ethanol, one or more sugar fermentation product, such as 2,3-butanediol, and/or a direct ethanol biomarker compared to the reference value is indicative that the patient will respond to therapy with an antibiotic and/or antifungal agent.

22. The method according to any one of aspects 20 or 21, the method comprising the step of predicting a response.

23. The method according to any one of aspects 20-22, wherein if the patient is predicted to be a responder, an antibiotic and/or antifungal agent is administered.

24. A method for identifying a patient having reduced liver-function or a patient at risk of reduced liver function that will respond to therapy with an antibiotic and/or antifungal agent, comprising the steps of:
a) establishing a reference value by measuring the concentration of ethanol, one or more sugar fermentation product, such as 2,3-butanediol, and/or a direct ethanol biomarker in a biological sample obtained from the patient before a carbohydrate has been administered;
b) administering a carbohydrate to the patient;
c) establishing a test value by measuring the concentration of ethanol, one or more sugar fermentation product, such as 2,3-butanediol, and/or a direct ethanol biomarker in a biological sample obtained from the patient after the carbohydrate has been administered; and
d) comparing the reference value to the test value, wherein the reference value is the concentration of ethanol, one or more sugar fermentation product, such as 2,3-butanediol, and/or a direct ethanol biomarker in a biological sample obtained from the patient before a carbohydrate has been administered;
   wherein the difference in the concentration of ethanol, one or more sugar fermentation product, such as 2,3-butanediol, and/or a direct ethanol biomarker in the test value compared to the concentration of ethanol, one or more sugar fermentation product, such as 2,3-butanediol, and/or a direct ethanol biomarker in the reference value is indicative that said patient will respond to therapy with an antibiotic and/or antifungal agent if said difference is above a threshold value.

25. A method of treating a patient with reduced liver function, the method comprising the steps of:
a) measuring the concentration of ethanol, one or more sugar fermentation product, such as 2,3-butanediol, and/or a direct ethanol biomarker in a biological sample obtained from the patient; and
b) predicting a response to treatment with an antibiotic and/or antifungal agent by comparing the concentration to a reference value from a subject with reduced liver function that does not respond to therapy with an antibiotic and/or antifungal agent and/or from a subject with reduced liver function that does respond to therapy with an antibiotic and/or antifungal agent; and/or from a healthy subject;
   wherein if the reference level is from a subject that does not respond to therapy with an antibiotic and/or antifungal agent, then an increase in the concentration of ethanol, one or more sugar fermentation product, such as 2,3-butanediol, and/or a direct ethanol biomarker compared to the reference value is indicative that the patient will respond to therapy with an antibiotic and/or antifungal agent;
   wherein if the reference level is from a subject that does respond to therapy with an antibiotic and/or antifungal agent, then the same or substantially the same or an increase in concentration of ethanol, one or more sugar fermentation product, such as 2,3-butanediol, and/or a direct ethanol biomarker is indicative that the patient will respond to therapy with an antibiotic and/or antifungal agent;
   or wherein if the reference level is from a healthy subject, then an increase in the concentration of ethanol, one or more sugar fermentation product, such as 2,3-butanediol, and/or a direct ethanol biomarker compared to the reference value is indicative that the patient will respond to therapy with an antibiotic and/or antifungal agent; and
c) if the patient is predicted to respond to therapy with an antibiotic and/or antifungal agent, administering an antibiotic and/or antifungal agent to the patient.

All of the features contained herein may be combined with any of the above aspects in any combination.

For a better understanding of the invention, and to show how embodiments of the same may be carried into effect, reference will now be made, by way of example, to the following examples.

### EXAMPLES

### Example 1

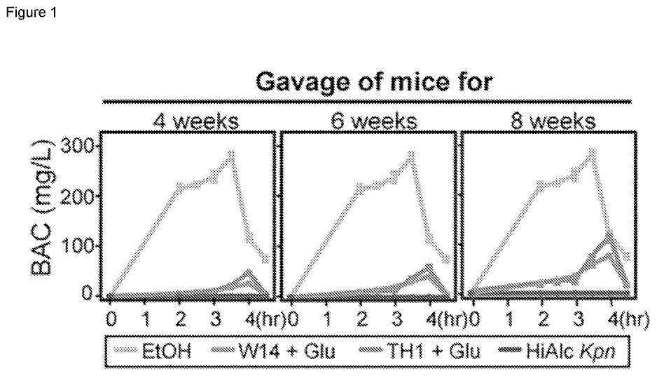

As shown in FIG. 1, mice treated with bacteria isolated from the gut of NASH patients have a spike in ethanol, as detected in blood, after glucose administration (Jing Yuan et al, Fatty Liver Disease Caused by High-Alcohol Producing Klebsiella pneumoniae, Cell Metabolism 30, 675-688, Oct. 1, 2019). This provides proof of concept that ethanol can be used as a biomarker to identify patients that will respond to therapy with an antibiotic.

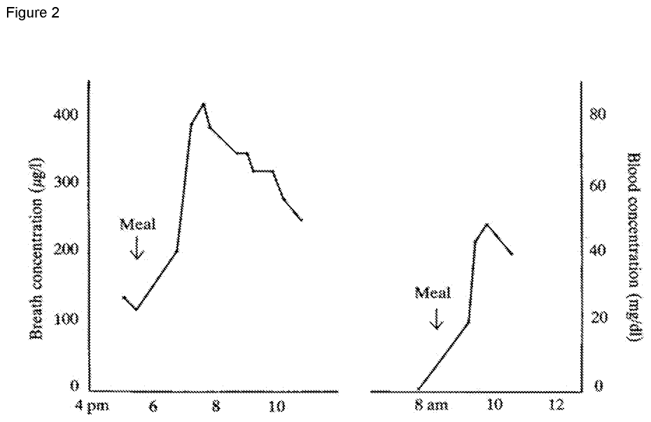

As shown in FIG. 2, a woman suffering from symptoms of ethanol intoxication, even if abstinent, showed a spike of levels of ethanol in breath and blood in the hours after a meal made of 78% carbohydrates. Candida albicans was found as the main agent producing ethanol. Treatment with an antifungal agent, carbohydrates restriction and use of laxatives led to disappearance of symptoms of alcohol intoxication. (H. Kaji et al, Intragastrointestinal Alcohol Fermentation Syndrome: Report of Two Cases and Review of the Literature, Journal of the Forensic Science Society, 24-5, 461-471, September 1984). This provides proof of concept that ethanol can be used as a biomarker to identify patients that will respond to therapy with an antifungal agent.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

Whereas particular examples of this invention have been described above for purposes of illustration, it will be evident to those skilled in the art that numerous variations of the details of the present invention may be made without departing from the invention as defined in the appended claims.

### Example 2

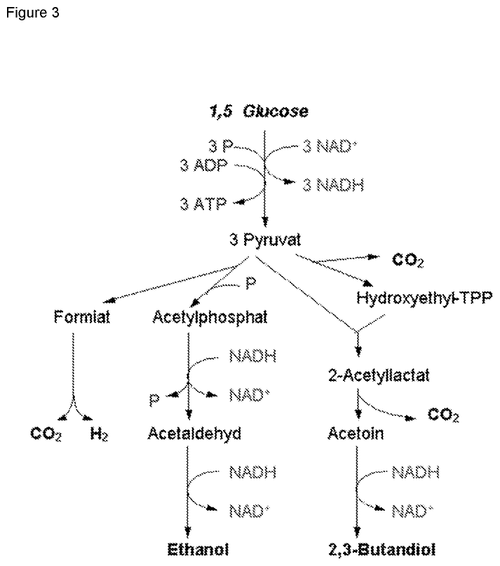

Metabolic fermentation of glucose generates several products as shown in FIG. 3. For example, Ethanol and 2-3-Butanediol exacerbate non-alcoholic steatohepatitis (1, 2).

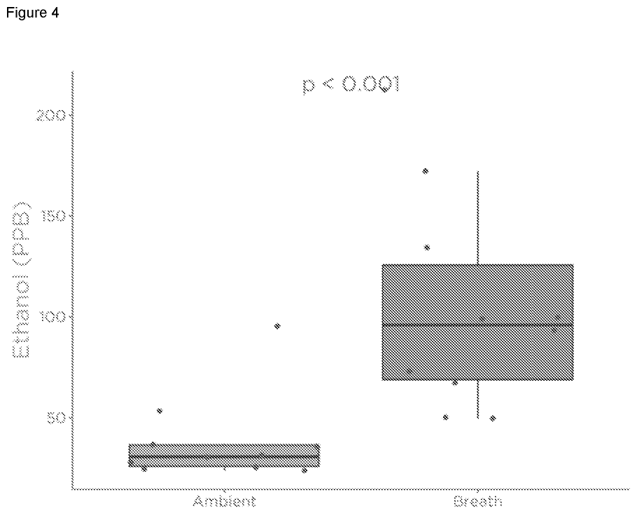

A total of 10 healthy subjects were recruited and instructed to fast overnight and avoid drinking alcohol the day before the experiment. Breath was analysed using selected ion flow tube mass spectrometry (SIFT-MS). A total of 3 consecutive breath samples were provided to measure baseline ethanol levels. Afterwards, 75 grams of glucose dissolved in 300 ml of water were ingested, and 3 consecutive breath samples were provided every 5 minutes for 90 minutes. Measured compounds were expressed as part per billion volume/volume (PPB) and plotted as a function of time. Ambient samples were collected before and after the procedure. As shown in FIG. 4 baseline breath samples showed higher level of ethanol in breath compared to ambient, indicating that ethanol is normally exhalated in small amount.

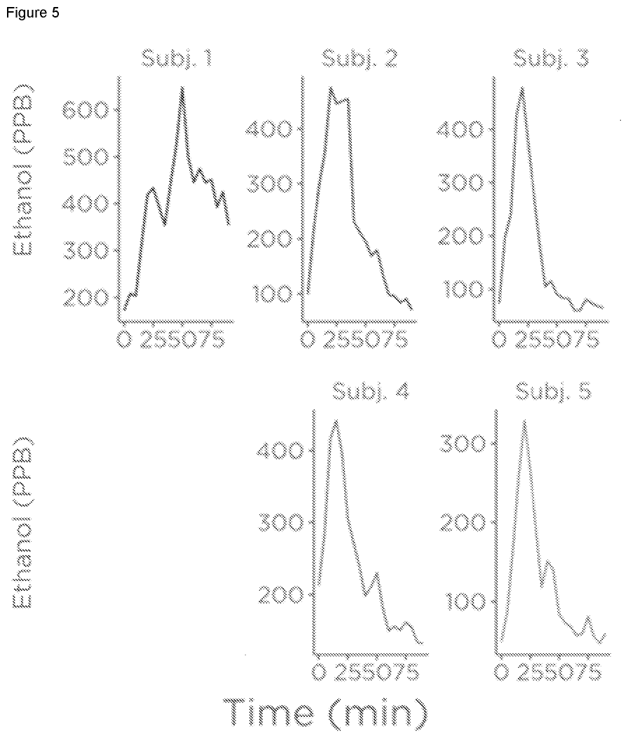

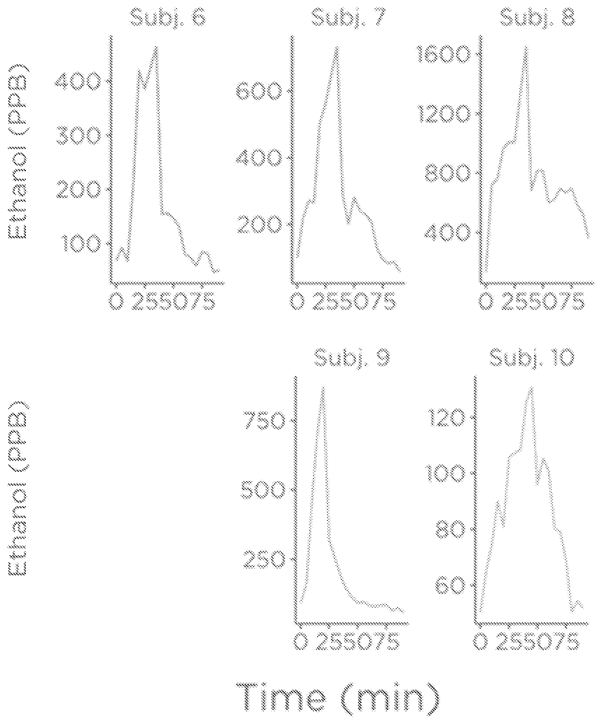

As shown in FIG. 5, glucose administration elicited a spike of ethanol in breath in all the tested subjects with a Cmax (maximum levels) with a median (M) of 400 PPB, and an interquartile range (IQR) of 303-593 PPB. The Tmax (time to maximum level) was achieved with a M of 27.5 min, and an IQR of 20-35 min. Levels returned to baseline at the end of the investigated timeframe for all but 1 subject. These results indicate that the used approach is suitable to measure variations of breath ethanol after carbohydrates intake.

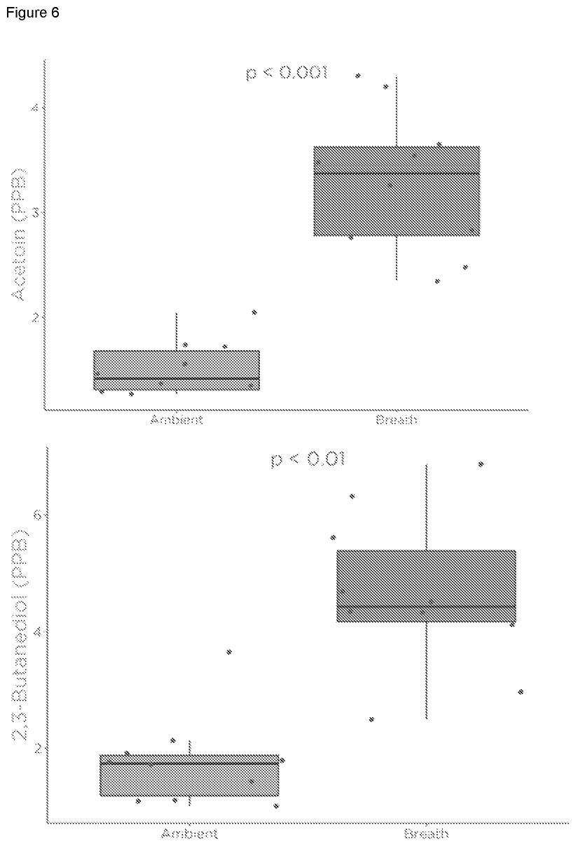

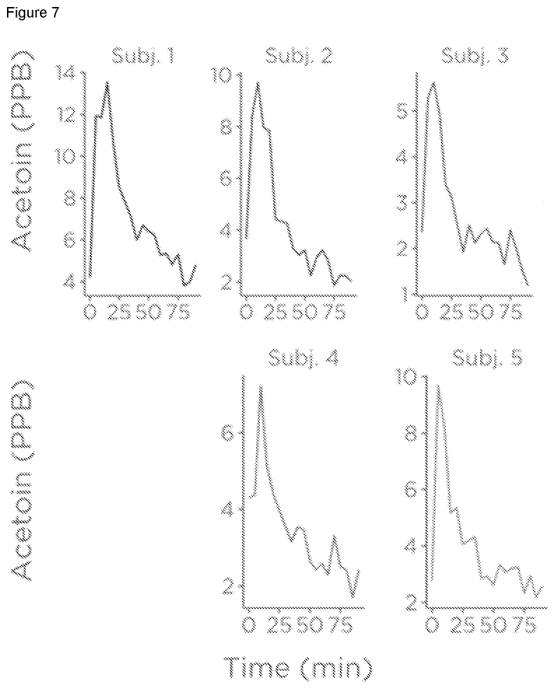

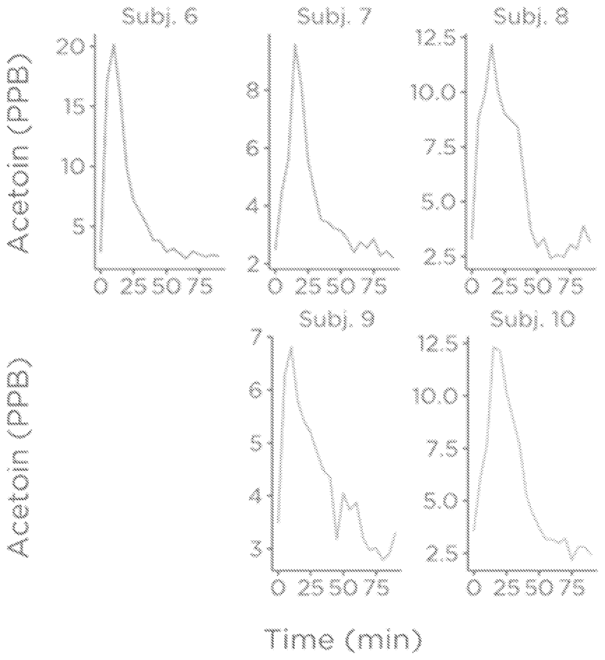

FIG. 6 shows baseline breath levels of acetoin and 2-3-butanediol. Glucose administration elicited a spike of acetoin in all the subjects with a Cmax (maximum levels) with a median (M) of 7 PPB, and an interquartile range (IQR) of 4-8.8 PPB. The Tmax (time to maximum level) was achieved with a M of 10 min, and an IQR of 10-15 min. Levels returned to baseline within 50 min for all investigated subjects (FIG. 7). No spike was observed for 2-3-Butanediol.

These results indicate that the used approach is suitable to measure variations of breath acetoin after carbohydrates intake and use this intermediate to evaluate the extent of 2-3-butanediol fermentation.

Methods:
Subjects characteristics:
Female = 7
Male = 3

| | Age | Weight | BMI |
|---|---|---|---|
| Min. | 25.00 | 52.00 | 19.1000 |
| 1st Qu. | 28.00 | 54.25 | 20.6650 |
| Median | 29.00 | 62.00 | 22.8750 |
| Mean | 33.50 | 64.60 | 22.7430 |
| 3rd Qu. | 37.25 | 70.25 | 24.9075 |
| Max. | 59.00 | 91.00 | 26.6700 |

All the procedures were approved by the “Reading Independent Ethics Committee” (RIEC) and all the subjects provided written informed consent forms. The subjects were asked to fast overnight and not drink alcohol the day before the experiment.

A total of 75 g of glucose from Bulk (https://www.bulk.com/uk/dextrose.html?view=ppc&pid=2378&gclid=CjwKCAjw-vmkBhBMEiwAlrMeF2SOAMhqgNq65EV8YC2XWu63e4ByfJckgwv5jKZfzCA6BbIB24sLSBoC—kQAvD_BwE) was weighted in a glass and 300 ml of water was added and mixed until the full amount was dissolved. A total of 6 blank samples were collected and analysed before and after the procedure. The subjects provided breath samples before and every 5 minutes over 90 minutes after glucose ingestion. Each timepoint was represented by a 15 second breath exhalation consecutively repeated 3 times ed o and averaged for every timepoint. Breath samples were directly sampled for Selected-ion flow-tube mass spectrometry (SIFT-MS). Subjects exhaled into a single use Mouthpiece and Reservoir Tube (PO007257 Imspex) that were replaced for each timepoint collection.

Scans were performed in batch mode; data were collected in single ion mode (SIM). SIM scan was 50 secs long. Detailed instrument settings are reported in table 2.

TABLE 2
SiFT-MS settings

| Analytes | Benzyl alcohol C7H8O m/z 108, limonene C10H16 m/z 136, 2-butanol C4H10O m/z 74, 2- | | | | | |
|---|---|---|---|---|---|---|
| | pentanol C5H12O m/z 88, 2-pentanone C5H10O m/z 86, 2,3-butanediol C4H10O2 m/z 90, | | | | | |
| | acetoin C4H8O2 m/z 88, acetone C3H6O m/z 58, benzaldehyde C7H6O m/z 106, benzoic | | | | | |
| | acid C7H6O2 m/z 122, 2-butanone C4H8O m/z 72, isoprene C5H8 m/z 68, trans-carveol | | | | | |
| | C10H16O m/z 152, octane C8H18 m/z 114, nonanal C9H18O m/z 142 | | | | | |
| Carrier | Helium | | | | | |
| gas | | | | | | |
| Flow rate | 20 ml/min | | | | | |
| Flow tube | Must match that used in the validation procedure (usually from ambient) | | | | | |
| pressure | | | | | | |
| Reagent | H₃O⁺ (19 m/z), H₃O⁺•(H₂O) (m/z 37), NO⁺ (m/z 30), O₂⁺ (m/z 32) | | | | | |
| ions | | | | | | |
| | NO⁺ | | H₃O⁺ | | O₂⁺ | |
| Product | 30.0 | NO+(0) | 19.0 | H3O+(0) | 32.0 | O2+(0) |
| ions | 48.0 | NO+(1) | 37.0 | H3O+•H20 | 59.0 | 2-butanol, |
| | | | | | | C3H7O+ |
| | 48.0 | NO+•(H2O)1 | 55.0 | H3O+•(H20)2 | 67.0 | Isoprene, |
| | | | | | | C5H7+ |
| | 68.0 | Isoprene, | 57.0 | 2-butanol, C4H9+ | 68.0 | Isoprene, |
| | | C5h8+ | | | | C5H8+ |
| | 73.0 | 2-butanol, | 59.0 | Acetone, C3H70+ | 72.0 | Butanone, |
| | | C4H9O+ | | | | C4H8O+ |
| | 87.0 | 2-pentanol, | 69.0 | Isoprene, | 86.0 | 2-pentanone, |
| | | C5H11O+ | | C5H8•H+ | | C5H10O+ |
| | 88.0 | Acetone, | 71.0 | 2-pentanol, | 88.0 | Acetonin, |
| | | NO+•C3H60 | | C5H11+ | | C4H8O2+ |
| | 89.0 | 2,3- | 73.0 | H3O+•(H2O)3 | 93.0 | Limonene, |
| | | butanediol, | | | | C7H9+ |
| | | C4H9O2+ | | | | |
| | 91.0 | Benzyl | 75.0 | 2-butanol, | 105.0 | Benzaldehyde, |
| | | alcohol, | | C4H11O+ | | C7H5O+ |
| | | C7H7+ | | | | |
| | 102.0 | Butanone, | 77.0 | Acetone, | 106.0 | Benzaldehyde, |
| | | NO+•C4H8O | | (CH3)2CO•H+•H2O | | C7H6O+ |
| | 108.0 | Benzyl | 87.0 | 2-pentanone, | 108.0 | Benzyl |
| | | alcohol, | | C5H11O+ | | alcohol, |
| | | C7H8O+ | | | | C7H8O+ |
| | 113.0 | Octane, | 113.0 | Octane, C8H17+ | 114.0 | Octane, |
| | | C8H17+ | | | | C8H18+ |
| | 116.0 | 2- | 123.0 | Benzoic acid, | 122.0 | Benzoic acid, |
| | | pentanone, | | C7H6O2•H+ | | C7H6O2+ |
| | | NO+• | | | | |
| | 118.0 | Acetoin, | 135.0 | Trans-carveol, | | |
| | | C4H8O2•NO+ | | C10H15+ | | |
| | 135.0 | Trans- | 137.0 | Limonene, | | |
| | | carveol, | | C10H17+ | | |
| | | C10H15+ | | | | |
| | 136.0 | Limonene, | 143.0 | Nonanal, | | |
| | | C10H16+ | | C9H19O+ | | |
| | 141.0 | Nonanal, | | | | |
| | | C9H17O+ | | | | |

| | |
|---|---|
| Possible | Mass 143 H3O+ has product conflict with nonanal, (benzaldehyde (secondary)) |
| conflicts | Mass 89 NO+ has product conflict with 2,3-butanediol, (2-pentanol (secondary)) |
| | Mass 135 NO+ has product conflict with trans-carveol, (limonene) |
| | Mass 68 O2+ has product conflict with isoprene, (limonene, nonanal) |
| | Mass 72 O2+ has product conflict with butanone, (nonanal) |
| | Mass 114 O2+ has product conflict with octane, (nonanal) |
| | All conflicts ions (greyed out) are removed due to small branching ratios (low |
| | proportions) as well as availability of other relevant unique product ions |
| Time/ion | 100 ms/100000 |
| count | |

### REFERENCES

1. Meijnikman A S, Davids M, Herrema H, Aydin O, Tremaroli V, Rios-Morales M, Levels H, et al. Microbiome-derived ethanol in nonalcoholic fatty liver disease. Nat Med 2022; 28:2100-2106.
2. Li N N, Li W, Feng J X, Zhang W W, Zhang R, Du S H, Liu S Y, et al. High alcohol-producing Klebsiella pneumoniae causes fatty liver disease through 2,3-butanediol fermentation pathway in vivo. Gut Microbes 2021; 13:1979883.

1. A method for identifying a patient having reduced liver-function or a patient at risk of reduced liver function that will respond to therapy with an antibiotic and/or antifungal agent comprising measuring the concentration of ethanol, one or more sugar fermentation product, such as 2,3-butanediol, and/or a direct ethanol biomarker in a biological sample from said patient, wherein the concentration of ethanol, one or more sugar fermentation product, such as 2,3-butanediol, and/or a direct ethanol biomarker is indicative that said patient will respond to therapy with an antibiotic and/or antifungal agent.

2. A method of predicting a response to an antibiotic and/or antifungal agent therapy in a patient having reduced liver-function or at risk of reduced liver function comprising measuring the concentration of ethanol, one or more sugar fermentation product, such as 2,3-butanediol, and/or a direct ethanol biomarker in a biological sample from said patient, wherein the concentration of ethanol, one or more sugar fermentation product, such as 2,3-butanediol, and/or a direct ethanol biomarker is indicative of a response or non-response of a patient to therapy with an antibiotic and/or antifungal agent.

3. The method of any one of claim 1 or 2, wherein the antibiotic and/or antifungal agent is an antibiotic.

4. The method according to any one of claims 1 to 3, comprising the step of: a) measuring the concentration of ethanol, one or more sugar fermentation product, such as 2,3-butanediol, and/or a direct ethanol biomarker in a biological sample obtained from the patient; and b) comparing the concentration to a reference value.

5. The method according to claim 4, wherein the reference value is from a subject with reduced liver function that does not respond to therapy with an antibiotic and/or antifungal agent; and wherein an increase in the concentration of ethanol, one or more sugar fermentation product, such as 2,3-butanediol, and/or a direct ethanol biomarker compared to the reference value is indicative that the patient will respond to therapy with an antibiotic and/or antifungal agent.

6. The method according to any one of claim 4 or 5, wherein the reference value is from a subject with reduced liver function that does respond to therapy with an antibiotic and/or antifungal agent; and wherein the same or substantially the same or an increase in concentration of ethanol, one or more sugar fermentation product, such as 2,3-butanediol, and/or a direct ethanol biomarker is indicative that the patient will respond to therapy with an antibiotic and/or antifungal agent.

7. The method according to any one of claims 4 to 6, and wherein the reference value is from a healthy subject; wherein an increase in the concentration of ethanol, one or more sugar fermentation product, such as 2,3-butanediol, and/or a direct ethanol biomarker compared to the reference value is indicative that the patient will respond to therapy with an antibiotic and/or antifungal agent.

8. The method according to any one of claims 1 to 3, comprising the steps of:
measuring the concentration of ethanol, one or more sugar fermentation product, such as 2,3-butanediol, and/or a direct ethanol biomarker in a biological sample obtained from the patient; and
comparing the concentration to a reference value from a subject with reduced liver function that does not respond to therapy with an antibiotic and/or antifungal agent; and/or from a subject with reduced liver function that does respond to therapy with an antibiotic and/or antifungal agent; and/or from a healthy subject;
wherein if the reference level is from a subject that does not respond to therapy with an antibiotic and/or antifungal agent, then an increase in the concentration of ethanol, one or more sugar fermentation product, such as 2,3-butanediol, and/or a direct ethanol biomarker compared to the reference value is indicative that the patient will respond to therapy with an antibiotic and/or antifungal agent;
wherein if the reference level is from a subject that does respond to therapy with an antibiotic and/or antifungal agent, then the same or substantially the same or an increase in concentration of ethanol, one or more sugar fermentation product, such as 2,3-butanediol, and/or a direct ethanol biomarker is indicative that the patient will respond to therapy with an antibiotic and/or antifungal agent; or
wherein if the reference level is from a healthy subject, then an increase in the concentration of ethanol, one or more sugar fermentation product, such as 2,3-butanediol, and/or a direct ethanol biomarker compared to the reference value is indicative that the patient will respond to therapy with an antibiotic and/or antifungal agent.

9. The method according to any preceding claim, wherein the direct ethanol biomarker is ethyl sulphate, ethyl-glucuronide, fatty acid ethyl esters, phosphatidylethanol, and/or combinations thereof.

10. The method according to any preceding claim wherein the sugar fermentation product is pyruvate, formic acid and/or ester derivates thereof, acetylphosphate, acetylaldehyde, hydroxyethyl-thiamine pyrophosphate (hydroxyethyl-TPP), acetyllactate, acetoin, 2,3-butanediol and/or combinations thereof, for example 2,3-butanediol.

11. The method according to any preceding claim, wherein the method comprises measuring the concentration of ethanol in a biological sample from said patient.

12. The method according to any preceding claim, wherein the patient has been diagnosed with a liver disease.

13. The method according to claim 12, wherein the patient has non-alcoholic fatty liver disease (NAFLD).

14. The method according to claim 12 or 13, wherein the patient has non-alcoholic steatohepatitis (NASH).

15. The method according to any one of claims 1 to 11, wherein the patient has not been diagnosed with a liver disease; and wherein the patient is at risk of reduced liver function.

16. The method according to claim 15, wherein the patient has one or more risk factors selected from obesity, alcoholism, family history of liver disease, type 2 diabetes, dyslipidaemia, and/or a genetic predisposition to a liver disease.

17. The method according to any preceding claim, further comprising the step of identifying a patient in need of treatment with an antibiotic and/or antifungal agent.

18. The method according to claim 17, further comprising administering an antibiotic and/or antifungal agent to said patient.

19. The method according to any preceding claim, further comprising the step of obtaining a biological sample from the patient.

20. The method according to any preceding claim, wherein the biological sample is hair, blood, stool, urine and/or exhaled breath.

21. The method according to claim 20, wherein the biological sample is blood, urine and/or exhaled breath.

22. The method according to claim 21, wherein the biological sample is exhaled breath.

23. The method according to any preceding claim, further comprising administering a carbohydrate to the patient.

24. The method according to any one of claims 19 to 22, further comprising administering a carbohydrate to the patient; wherein the step of administering a carbohydrate to the patient is carried out prior to the step of obtaining a biological sample from the patient.

25. The method according to any of claim 23 or 24, wherein the carbohydrate is labelled such that at least a portion of the ethanol, one or more sugar fermentation product, such as 2,3-butanediol, or a direct ethanol biomarker is labelled.

26. The method according to any one of claims 11 to 25, wherein the method comprises measuring the concentration of ethanol in a biological sample obtained from the patient and at least a portion of the ethanol detected is labelled.

27. The method according to any one of claim 25 or 26, wherein the label is 12C, 13C, 14C, 2H, 17O and/or 18O.

28. The method according to claim 27, wherein the label is 13C.

29. The method according to any of claims 23 to 28, wherein the step of administering a carbohydrate to the patient is carried out at least 10 minutes, such as at least 20, 30, 40, 50, 60, 90, 120 or 180 minutes, prior to the step of obtaining a biological sample from the patient.

30. The method according to any preceding claim, further comprising the step of administering an alcohol dehydrogenase (ADH) inhibitor to the patient.

31. The method according to any one of claims 23 to 30, further comprising the step of administering an alcohol dehydrogenase (ADH) inhibitor to the patient; wherein the step of administering an ADH inhibitor to the patient is carried out prior to the step of obtaining a biological sample from the patient.

32. The method according to any one of claim 30 or 31, wherein where the method includes the step of administering a carbohydrate to a patient, the step of administering an ADH inhibitor to the patient is carried out before and/or after the step of administering a carbohydrate to a patient.

33. The method according to any preceding claim, wherein the biological sample is blood and a concentration of ethanol, one or more sugar fermentation product, such as 2,3-butanediol, and/or a direct ethanol biomarker of at least 20 mmol/L is indicative that said patient will respond to therapy with an antibiotic and/or antifungal agent.

34. The method according to any preceding claim, wherein the biological sample is exhaled breath and a concentration of ethanol, one or more sugar fermentation product, such as 2,3-butanediol, and/or a direct ethanol biomarker of at least 0.0025 mmol/L is indicative that said patient will respond to therapy with an antibiotic and/or antifungal agent.

35. The method according to claim 34, wherein a concentration of ethanol, one or more sugar fermentation product, such as 2,3-butanediol, and/or a direct ethanol biomarker of at least 0.005 mmol/L is indicative that said patient will respond to therapy with an antibiotic and/or antifungal agent.

36. The method according to claim 35, wherein a concentration of ethanol, one or more sugar fermentation product, such as 2,3-butanediol, and/or a direct ethanol biomarker of at least 0.01 mmol/L is indicative that said patient will respond to therapy with an antibiotic and/or antifungal agent.

37. The method according to any preceding claim, wherein the ethanol, one or more sugar fermentation product, such as 2,3-butanediol, and/or a direct ethanol biomarker is labelled.

38. The method according to claim 37, wherein the presence of labelled ethanol, one or more sugar fermentation product, such as 2,3-butanediol, and/or a direct ethanol biomarker in the biological sample from the patient is indicative that said patient will respond to therapy with an antibiotic and/or antifungal agent.

39. The method according to any preceding claim, wherein the antibiotic comprises rifaximin; neomycin; metronidazole; flouroquinolones; cephalosporins; trimethoprim-sulfamethoxazole; flucloxacillin; cephalothin; aztreonam; cabimicina; and combinations thereof; and/or wherein the antifungal agent comprises clotrimazole, econazole, miconazole, terbinafine, fluconazole, ketoconazole, nystatin, amphotericin, and/or combinations thereof.

40. The method according to claim 39, wherein the flouroquinolones comprise norfloxacin and/or ciprofloxacin.

41. The method according to any one of claim 39 or 40, wherein the cephalosporins comprises ceftriaxone and/or cefotaxime.

42. A method of treating a patient with reduced liver function, the method comprising the steps of:
a) measuring the concentration of ethanol, one or more sugar fermentation product, such as 2,3-butanediol, and/or a direct ethanol biomarker in a biological sample obtained from the patient; and
b) predicting a response to treatment with an antibiotic and/or antifungal agent by comparing the concentration to a reference value from a subject with reduced liver function that does not respond to therapy with an antibiotic and/or antifungal agent; and/or from a subject with reduced liver function that does respond to therapy with an antibiotic and/or antifungal agent; and/or from a healthy subject;
wherein if the reference level is from a subject that does not respond to therapy with an antibiotic and/or antifungal agent, then an increase in the concentration of ethanol, one or more sugar fermentation product, such as 2,3-butanediol, and/or a direct ethanol biomarker compared to the reference value is indicative that the patient will respond to therapy with an antibiotic and/or antifungal agent;
wherein if the reference level is from a subject that does respond to therapy with an antibiotic and/or antifungal agent, then the same or substantially the same or an increase in concentration of ethanol, one or more sugar fermentation product, such as 2,3-butanediol, and/or a direct ethanol biomarker is indicative that the patient will respond to therapy with an antibiotic and/or antifungal agent; or wherein if the reference level is from a healthy, then an increase in the concentration of ethanol, one or more sugar fermentation product, such as 2,3-butanediol, and/or a direct ethanol biomarker compared to the reference value is indicative that the patient will respond to therapy with an antibiotic and/or antifungal agent; and
c) if the patient is predicted to respond to therapy with an antibiotic and/or antifungal agent, administering an antibiotic and/or antifungal agent to the patient.

43. The method according to claim 42, wherein the antibiotic and/or antifungal agent is an antibiotic.

44. The method according to any one of claim 42 or 43, wherein the direct ethanol biomarker is ethyl sulphate, ethyl-glucuronide, fatty acid ethyl esters, phosphatidylethanol, and/or combinations thereof.

45. The method according to any one of claims 42 to 44, wherein the sugar fermentation product is pyruvate, formic acid and/or ester derivates thereof, acetylphosphate, acetylaldehyde, hydroxyethyl-thiamine pyrophosphate (hydroxyethyl-TPP), acetyllactate, acetoin, 2,3-butanediol and/or combinations thereof, for example 2,3-butanediol.

46. The method according to any one of claims 42 to 45, wherein the method comprises measuring the concentration of ethanol in a biological sample from said patient.

47. The method according to any one of claims 42 to 46, wherein the patient has been diagnosed with a liver disease.

48. The method according to claim 47, wherein the patient has non-alcoholic fatty liver disease (NAFLD).

49. The method according to any one of claim 47 or 48, wherein the patient has non-alcoholic steatohepatitis (NASH).

50. The method according to any one of claims 42 to 49, further comprising the step of obtaining a biological sample from the patient.

51. The method according to any one of claims 42 to 50, wherein the biological sample is hair, blood, stool, urine and/or exhaled breath.

52. The method according to claim 51, wherein the biological sample is blood, urine and/or exhaled breath.

53. The method according to claim 52, wherein the biological sample is exhaled breath.

54. The method according to any one of claims 47 to 53, further comprising administering a carbohydrate to the patient.

55. The method according to any one of claims 42 to 53, further comprising administering a carbohydrate to the patient; wherein the step of administering a carbohydrate to the patient is carried out prior to the step of obtaining a biological sample from the patient.

56. The method according to any one of claim 54 or 55, wherein the carbohydrate is labelled such that at least a portion of the ethanol, one or more sugar fermentation product, such as 2,3-butanediol and/or a direct ethanol biomarker is labelled.

57. The method according to any one of claims 46 to 56, wherein the method comprises measuring the concentration of ethanol in a biological sample obtained from the patient and at least a portion of the ethanol detected is labelled.

58. The method according to any one of claim 56 or 57, wherein the label is 12C, 13C, 14C, 2H, 17O and/or 18O.

59. The method according to claim 58, wherein the label is 13C.

60. The method according to any of claims 54 to 59, wherein the step of administering a carbohydrate to the patient is carried out at least 10 minutes, such as at least 20, 30, 40, 50, 60, 90, 120 or 180 minutes prior to the step of obtaining a biological sample from the patient.

61. The method according to any one of claims 42 to 60, further comprising the step of administering an alcohol dehydrogenase (ADH) inhibitor to the patient.

62. The method according to any one of claims 42 to 61, further comprising the step of administering an alcohol dehydrogenase (ADH) inhibitor to the patient; wherein the step of administering an ADH inhibitor to the patient is carried out prior to the step of obtaining a biological sample from the patient.

63. The method according to any one of claim 61 or 62, wherein where the method includes the step of administering a carbohydrate to a patient, the step of administering an ADH inhibitor to the patient is carried out before and/or after the step of administering a carbohydrate to a patient.

64. The method according to any one of claims 42 to 63, wherein the biological sample is blood and a concentration of ethanol, one or more sugar fermentation product, such as 2,3-butanediol, and/or a direct ethanol biomarker of at least 20 mmol/L is indicative that said patient will respond to therapy with an antibiotic and/or antifungal agent.

65. The method according to any one of claims 42 to 64, wherein the biological sample is exhaled breath and a concentration of ethanol, one or more sugar fermentation product, such as 2,3-butanediol, and/or a direct ethanol biomarker of at least 0.0025 mmol/L is indicative that said patient will respond to therapy with an antibiotic and/or antifungal agent.

66. The method according to claim 65, wherein a concentration of ethanol, one or more sugar fermentation product, such as 2,3-butanediol, and/or a direct ethanol biomarker of at least 0.005 mmol/L is indicative that said patient will respond to therapy with an antibiotic and/or antifungal agent.

67. The method according to claim 66, wherein a concentration of ethanol, one or more sugar fermentation product, such as 2,3-butanediol, and/or a direct ethanol biomarker of at least 0.01 mmol/L is indicative that said patient will respond to therapy with an antibiotic and/or antifungal agent.

68. The method according to any one of claims 42 to 67, wherein the ethanol, one or more sugar fermentation product, such as 2,3-butanediol, and/or a direct ethanol biomarker is labelled.

69. The method according to claim 68, wherein the presence of labelled ethanol, one or more sugar fermentation product, such as 2,3-butanediol, and/or a direct ethanol biomarker in the biological sample from the patient is indicative that said patient will respond to therapy with an antibiotic and/or antifungal agent.

70. The method according to any one of claims 42 to 69, wherein the antibiotic comprises rifaximin; neomycin; metronidazole; flouroquinolones; cephalosporins; trimethoprim-sulfamethoxazole; flucloxacillin; cephalothin; aztreonam; cabimicina; and combinations thereof; and/or wherein the antifungal agent comprises clotrimazole, econazole, miconazole, terbinafine, fluconazole, ketoconazole, nystatin, amphotericin, and/or combinations thereof.

71. The method according to claim 70, wherein the flouroquinolones comprise norfloxacin and/or ciprofloxacin.

72. The method according to any one of claim 70 or 71, wherein the cephalosporins comprises ceftriaxone and/or cefotaxime.

73. A method for identifying a patient having reduced liver-function or a patient at risk of reduced liver function that will respond to therapy with an antibiotic and/or antifungal agent, comprising the steps of:
a) establishing a reference value by measuring the concentration of ethanol, one or more sugar fermentation product, such as 2,3-butanediol, and/or a direct ethanol biomarker in a biological sample obtained from the patient before a carbohydrate has been administered;
b) administering a carbohydrate to the patient;
c) establishing a test value by measuring the concentration of ethanol, one or more sugar fermentation product, such as 2,3-butanediol, and/or a direct ethanol biomarker in a biological sample obtained from the patient after the carbohydrate has been administered; and
d) comparing the reference value to the test value, wherein the reference value is the concentration of ethanol, one or more sugar fermentation product, such as 2,3-butanediol, and/or a direct ethanol biomarker in a biological sample obtained from the patient before a carbohydrate has been administered;
wherein the difference in the concentration of ethanol, one or more sugar fermentation product, such as 2,3-butanediol, and/or a direct ethanol biomarker in the test value compared to the concentration of ethanol, one or more sugar fermentation product, such as 2,3-butanediol, and/or a direct ethanol biomarker in the reference value is indicative that said patient will respond to therapy with an antibiotic antifungal agent if said difference is above a threshold value.

74. The method according to claim 73, wherein the antibiotic and/or antifungal agent is an antibiotic.

75. The method according to any one of claim 73 or 74, wherein the direct ethanol biomarker is ethyl sulphate, ethyl-glucuronide, fatty acid ethyl esters, phosphatidylethanol, and/or combinations thereof.

76. The method according to any one of claims 73 to 75, wherein the sugar fermentation product is pyruvate, formic acid and/or ester derivates thereof, acetylphosphate, acetylaldehyde, hydroxyethyl-thiamine pyrophosphate (hydroxyethyl-TPP), acetyllactate, acetoin, 2,3-butanediol and/or combinations thereof, for example 2,3-butanediol.

77. The method according to any one of claims 73 to 76, wherein the method comprises measuring the concentration of ethanol in a biological sample from said patient.

78. The method according to any one of claims 73 to 77, wherein the patient has been diagnosed with a liver disease.

79. The method according to claim 78, wherein the patient has non-alcoholic fatty liver disease (NAFLD).

80. The method according to claim 78 or 79, wherein the patient has non-alcoholic steatohepatitis (NASH).

81. The method according to any one of claims 73 to 77, wherein the patient has not been diagnosed with a liver disease; and wherein the patient is at risk of reduced liver function.

82. The method according to claim 81, wherein the patient has one or more risk factors selected from obesity, alcoholism, family history of liver disease, type 2 diabetes, dyslipidaemia, and/or a genetic predisposition to a liver disease.

83. The method according to any one of claims 73 to 82, further comprising the step of identifying a patient in need of treatment with an antibiotic and/or antifungal agent.

84. The method according to claim 83, further comprising administering an antibiotic and/or antifungal agent to said patient.

85. The method according to any one of claims 73 to 84, comprising obtaining biological samples from the patient.

86. The method according to any one of claims 73 to 85, wherein the biological sample is hair, blood, stool, urine and/or exhaled breath.

87. The method according to claim 86, wherein the biological sample is blood, urine and/or exhaled breath.

88. The method according to claim 87, wherein the biological sample is exhaled breath.

89. The method according to any one of claims 73 to 88, wherein the carbohydrate is labelled such that at least a portion of the ethanol, one or more sugar fermentation product, such as 2,3-butanediol, and/or a direct ethanol biomarker is labelled.

90. The method according to any one of claims 77 to 89, wherein the method comprises measuring the concentration of ethanol in a biological sample obtained from the patient and at least a portion of the ethanol detected is labelled.

91. The method according to any one of claim 89 or 90, wherein the label is 12C, 13C, 14C, 2H, 17O and/or 18O.

92. The method according to claim 91, wherein the label is 13C.

93. The method according to any of claims 73 to 92, wherein the step of administering a carbohydrate to the patient is carried out at least 10 minutes, such as at least 20, 30, 40, 50, 60, 90, 120 or 180 minutes, prior to the step of obtaining a biological sample from the patient in step c).

94. The method according to any one of claims 73 to 93, further comprising the step of administering an alcohol dehydrogenase (ADH) inhibitor to the patient.

95. The method according to any one of claims 85 to 94, further comprising the step of administering an alcohol dehydrogenase (ADH) inhibitor to the patient; wherein the step of administering an ADH inhibitor to the patient is carried out prior to the step of obtaining a biological sample from the patient.

96. The method according to any one of claim 94 or 95, wherein the step of administering an ADH inhibitor to the patient is carried out before and/or after the step of administering a carbohydrate to a patient.

97. The method according to any one of claims 73 to 96, wherein the biological sample is blood and the threshold is at least 20 mmol/L.

98. The method according to any one of claims 73 to 97, wherein the biological sample is exhaled breath and the threshold value is at least 0.0025 mmol/L.

99. The method according to claim 98, wherein a concentration of ethanol, one or more sugar fermentation product, such as 2,3-butanediol, and/or a direct ethanol biomarker of at least 0.005 mmol/L is indicative that said patient will respond to therapy with an antibiotic and/or antifungal agent.

100. The method according to claim 99, wherein the threshold is at least 0.01 mmol/L.

101. The method according to any one of claims 73 to 100, wherein the ethanol, one or more sugar fermentation product, such as 2,3-butanediol, and/or a direct ethanol biomarker is labelled.

102. The method according to any one of claims 73 to 101, wherein the antibiotic comprises rifaximin; neomycin; metronidazole; flouroquinolones; cephalosporins; trimethoprim-sulfamethoxazole; flucloxacillin; cephalothin; aztreonam; cabimicina; and combinations thereof; and/or wherein the antifungal agent comprises clotrimazole, econazole, miconazole, terbinafine, fluconazole, ketoconazole, nystatin, amphotericin, and/or combinations thereof.

103. The method according to claim 102, wherein the flouroquinolones comprise norfloxacin and/or ciprofloxacin.

104. The method according to any one of claim 102 or 103, wherein the cephalosporins comprises ceftriaxone and/or cefotaxime.

105. A method of treating a patient with reduced liver function, the method comprising the steps of:
a) establishing a reference value by measuring the concentration of ethanol, one or more sugar fermentation product, such as 2,3-butanediol, and/or a direct ethanol biomarker in a biological sample obtained from the patient before a carbohydrate has been administered;
b) administering a carbohydrate to the patient;
c) establishing a test value by measuring the concentration of ethanol, one or more sugar fermentation product, such as 2,3-butanediol, and/or a direct ethanol biomarker in a biological sample obtained from the patient after the carbohydrate has been administered;
d) comparing the reference value to the test value, wherein the reference value is the concentration of ethanol, one or more sugar fermentation product, such as 2,3-butanediol, and/or a direct ethanol biomarker in a biological sample obtained from the patient before a carbohydrate has been administered, and wherein the difference in the concentration of ethanol, one or more sugar fermentation product, such as 2,3-butanediol, and/or a direct ethanol biomarker in the test value compared to the concentration of ethanol, one or more sugar fermentation product, such as 2,3-butanediol, and/or a direct ethanol biomarker in the reference value is indicative that said patient will respond to therapy with an antibiotic and/or antifungal agent if said difference is above a threshold value; and
e) if the patient is predicted to respond to therapy with an antibiotic and/or antifungal agent, administering an antibiotic and/or antifungal agent to the patient.

106. The method according to claim 105, wherein the antibiotic and/or antifungal agent is an antibiotic.

107. The method according to any one of claim 106 or 107, wherein the direct ethanol biomarker is ethyl sulphate, ethyl-glucuronide, fatty acid ethyl esters, phosphatidylethanol, and/or combinations thereof.

108. The method according to any one of claims 105 to 107, wherein the sugar fermentation product is pyruvate, formic acid and/or ester derivates thereof, acetylphosphate, acetylaldehyde, hydroxyethyl-thiamine pyrophosphate (hydroxyethyl-TPP), acetyllactate, acetoin, 2,3-butanediol and/or combinations thereof, for example 2,3-butanediol.

109. The method according to any one of claims 105 to 107, wherein the method comprises measuring the concentration of ethanol in a biological sample from said patient.

110. The method according to any one of claims 105 to 109, wherein the patient has been diagnosed with a liver disease.

111. The method according to claim 110, wherein the patient has non-alcoholic fatty liver disease (NAFLD).

112. The method according to any one of claim 110 or 111, wherein the patient has non-alcoholic steatohepatitis (NASH).

113. The method according to any one of claims 105 to 112, further comprising obtaining biological samples from the patient.

114. The method according to any one of claims 105 to 113, wherein the biological sample is hair, blood, stool, urine and/or exhaled breath.

115. The method according to claim 114, wherein the biological sample is blood, urine and/or exhaled breath.

116. The method according to claim 115, wherein the biological sample is exhaled breath.

117. The method according to any one of claims 105 to 116, wherein the carbohydrate is labelled such that at least a portion of the ethanol, one or more sugar fermentation product, such as 2,3-butanediol, and/or a direct ethanol biomarker is labelled.

118. The method according to any one of claims 109 to 117, wherein the method comprises measuring the concentration of ethanol in a biological sample obtained from the patient and at least a portion of the ethanol detected is labelled.

119. The method according to any one of claim 117 or 118, wherein the label is 12C, 13C, 14C, 2H, 17O and/or 18O.

120. The method according to claim 119, wherein the label is 13C.

121. The method according to any of claims 113 to 120, wherein the step of administering a carbohydrate to the patient is carried out at least 10 minutes, such as at least 20, 30, 40, 50, 60, 90, 120 or 180 minutes, prior to the step of obtaining a biological sample from the patient in step c).

122. The method according to any one of claims 105 to 121, further comprising the step of administering an alcohol dehydrogenase (ADH) inhibitor to the patient.

123. The method according to claim 122, wherein the step of administering an ADH inhibitor to the patient is carried out before and/or after the step of administering a carbohydrate to a patient.

124. The method according to any one of claims 105 to 123, wherein the biological sample is blood and the threshold value is at least 20 mmol/L.

125. The method according to any one of claims 105 to 124, wherein the biological sample is exhaled breath and the threshold value is at least 0.0025 mmol/L.

126. The method according to claim 125, wherein the biological sample is exhaled breath and the threshold value is at least 0.005 mmol/L.

127. The method according to claim 126, wherein the threshold value is at least 0.01 mmol/L.

128. The method according to any one of claims 105 to 127, wherein the ethanol, one or more sugar fermentation product, such as 2,3-butanediol, and/or a direct ethanol biomarker is labelled.

129. The method according to claim 128, wherein the presence of labelled ethanol, one or more sugar fermentation product, such as 2,3-butanediol, and/or a direct ethanol biomarker in the biological sample from the patient is indicative that said patient will respond to therapy with an antibiotic and/or antifungal agent.

130. The method according to any one of claims 105 to 129, wherein the antibiotic comprises rifaximin; neomycin; metronidazole; flouroquinolones; cephalosporins; trimethoprim-sulfamethoxazole; flucloxacillin; cephalothin; aztreonam; cabimicina; and combinations thereof; and/or wherein the antifungal agent comprises clotrimazole, econazole, miconazole, terbinafine, fluconazole, ketoconazole, nystatin, amphotericin, and/or combinations thereof.

131. The method according to claim 130, wherein the flouroquinolones comprise norfloxacin and/or ciprofloxacin.

132. The method according to any one of claim 130 or 131, wherein the cephalosporins comprises ceftriaxone and/or cefotaxime.
